(19) United States
(12) Patent Application Publication
(10) Pub. No.: US 2024/0115118 A1
(43) Pub. Date: Apr. 11, 2024

(54) PULL WIRE TENSIONING MECHANISMS FOR ENDOSCOPES

(71) Applicant: GYRUS ACMI, INC. D/B/A OLYMPUS SURGICAL TECHNOLOGIES AMERICA, Westborough, MA (US)

(72) Inventors: Robert E. Ailinger, Norwood, MA (US); Kelly David Christensen, Centerville, UT (US); Justin Garth Tidwell, North Salt Lake, UT (US); Derek Alder Hutchison, Vineyard, UT (US); Spence Edward Cornia, Sandy, UT (US); Dustin Edward Gorringe, Riverton, UT (US); Jeffrey C. Smith, Poland, IN (US)

(21) Appl. No.: 18/306,834

(22) Filed: Apr. 25, 2023

Related U.S. Application Data
(60) Provisional application No. 63/378,406, filed on Oct. 5, 2022.

(51) Int. Cl. A61B 1/005; A61B 1/00

(52) U.S. Cl. CPC A61B 1/0057; A61B 1/00128; A61B 1/0052

(57) ABSTRACT

An endoscope comprises a handpiece housing, an elongate flexible shaft extending from the handpiece housing, a pull wire extending from the handpiece housing into the elongate flexible shaft, and a tensioning mechanism configured to adjust tension in the pull wire. A method for adjusting tension in a pull wire of a controller for an endoscope comprises preparing an endoscope for use before a procedure, adjusting tension in a pull wire for deflecting a flexible elongate shaft of the endoscope to reduce knob dwell, and performing an endoscopic procedure with the endoscope.

### PRIORITY CLAIM

This application claims the benefit of priority to U.S. Provisional Patent Application Ser. No. 63/378,406, filed Oct. 5, 2022, the contents of which are incorporated by reference in their entirety.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices, such as endoscopes, having a controller connected to an elongate body to adjust pull wires that extend through the elongate body.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path. For example, duodenoscopy procedures (e.g., Endoscopic Retrograde Cholangio-Pancreatography, hereinafter “ERCP” procedures) involve the use of an auxiliary scope (also referred to as a daughter scope or cholangioscope) that can be advanced through the working channel of a main scope (also referred to as a mother scope or duodenoscope). Furthermore, another device (e.g., a treatment device), such as a tissue retrieval device used for biopsies, can be inserted into the auxiliary scope. As such, the treatment device can be controlled and guided via pushing and pulling of the shafts of the main scope and auxiliary scope, such as via the use of pull wires extending within the shafts of the main scope and auxiliary scope. The pull wires are typically anchored at a distal end of the shaft, connected to a controller at a proximal end of the shaft, and freely slidable within the shaft therebetween. Operation of a knob or lever on the controller can cause the pull wire to induce bending of the shaft. Typically, pull wires are arranged in pairs to produce opposite bending of the shaft.

### SUMMARY

The present disclosure recognizes that problems to be solved with conventional medical devices, and in particular endoscopes and duodenoscopes, include, among other things, the undesirability of elongate insertion shafts of endoscopes shrinking due to various factors. For example, shafts of endoscopes are typically made of a polymer material. Such material can shrink due to various factors, including environmental conditions, transportation conditions and aging. In particular, insertion shafts can be subject to sterilization procedures that occur at elevated temperatures. In examples, shafts of endoscopes can shrink about three to four millimeters. The resulting shrinkage of the insertion shafts can result in knob dwell. Knob dwell is the presence of slack in pull wires configured to bend the distal end of the elongate insertion shaft. The slack results in control features for the pull wires, e.g., knobs, having a certain amount of unresponsiveness. For example, a knob can be rotated without the distal end of the elongate insertion shaft bending while slack in the operative pull wire is being taken out. After the slack is removed, rotation of the knob in the same direction will produce the desired pulling on the operative pull wire. However, rotation of the knob in the opposite direction will require slack in the opposing pull wire to be taken up before responsiveness in achieved. This process is repeated each time the knob is rotated in opposite directions. Thus, knob dwell produces an undesirable user experience.

The present disclosure can help provide solutions to these and other problems by providing systems, devices and methods for reducing or eliminating slack in pull wires, particularly for slack arising after manufacturing and or sterilization due to shrinkage in axial length of the elongate insertion shaft.

In examples, the present disclosure includes multiple devices and associated methods for displacing an elongate insertion shaft relative to a control handle to remove slack from pull wires within the elongate insertion shaft.

In additional examples, the present disclosure includes multiple devices and associated methods for displacing pull wires relative to an elongate insertion shaft to remove slack from pull wires within the elongate insertion shaft.

In examples, an endoscope comprises a handpiece housing, an elongate flexible shaft extending from the handpiece housing, a pull wire extending from the handpiece housing into the elongate flexible shaft, and a tensioning mechanism configured to adjust tension in the pull wire.

In examples, a method for adjusting tension in a pull wire of a controller for an endoscope comprises preparing an endoscope for use before a procedure, adjusting tension in a pull wire for deflecting a flexible elongate shaft of the endoscope to reduce knob dwell, and performing an endoscopic procedure with the endoscope.

### BRIEF DESCRIPTION OF DRAWINGS

FIG. 1 is a schematic diagram of an endoscopy system comprising an imaging and control system and an endoscope, such as duodenoscope, with which the tensioning devices of the present disclosure can be used.

FIG. 2 is a schematic diagram of the imaging and control system of FIG. 1 showing the imaging and control system connected to the endoscope.

FIG. 3 is an exploded view of a handle section of an endoscope and a shaft including a first example of a tensioning mechanism according to the present disclosure.

FIG. 4A is a side view of the tensioning mechanism of FIG. 3 with the shaft in a retracted state.

FIG. 4B is a side view of the tensioning mechanism of FIG. 3 with the shaft in an extended state.

FIG. 5 is top view of the tensioning mechanism of FIGS. 3-4B showing geometry of a rotary tensioning mechanism.

FIGS. 6A and 6B are side views of a tensioning mechanism comprising a spring and a removable pin.

FIGS. 7A and 7B are side views of a spring-loaded tensioning mechanism in retracted and advanced states, respectively.

FIG. 8 is a perspective view of a tensioning mechanism comprising a ratchet mechanism.

FIG. 9 is a perspective view of a tensioning mechanism comprising an internal screw mechanism with a lever.

FIGS. 10A and 10B are external and internal views of a tensioning mechanism comprising an internal screw mechanism with a knob.

FIG. 11 is a schematic cross-sectional view of a tensioning mechanism comprising a button-activated wedge.

FIGS. 12A and 12B are schematic cross-sectional views of a spring-loaded tensioning mechanism for pull wires in a standby state and an activated state, respectively.

FIG. 12C is a side cross-sectional view of a tensioner of the spring-loaded tensioning mechanism of FIGS. 12A and 12B showing a pull wire seated in a trough.

FIGS. 13A and 13B are schematic cross-sectional views of a torsion spring tensioning mechanism for pull wires in a standby state and an activated state, respectively.

FIG. 13C is a schematic top view of the torsion spring tensioning mechanism of FIGS. 13A and 13B.

FIGS. 14A and 14B are schematic cross-sectional views of a rotatable tensioning mechanism for pull wires in a standby state and an activated state, respectively.

FIG. 15A is a perspective view of a rotatable barrel tensioning mechanism of the present disclosure comprising a knob component and a barrel component.

FIGS. 15B and 15C are perspective views of a first side and a second side of the knob component of FIG. 15A, respectively.

FIGS. 15D and 15E are perspective views of a first side and a second side of the barrel component of FIG. 15A, respectively.

FIG. 15F is a perspective view of the rotatable barrel tensioning mechanism of FIG. 15A in a standby state.

FIG. 15G is a perspective view of the rotatable barrel tensioning mechanism of FIG. 15A in an intermediate state.

FIG. 15H is a perspective view of the rotatable barrel tensioning mechanism of FIG. 15A in an activated state.

FIGS. 16A and 16B are schematic cross-sectional view of a rack and pinion tensioning mechanism for pull wires in a standby state and an activated state, respectively.

FIGS. 17A, 17B and 17C show a retention strap for an auxiliary scope being wrapped around a primary scope to actuate a tensioning mechanism of the present disclosure.

FIG. 18 is a schematic illustration of the auxiliary scope of FIGS. 17A-17B positioned within a packaging container.

FIG. 19 is a block diagram illustrating operations of various methods for reducing slack in pull wires to, for example, reduce knob dwell.

FIG. 20 is a flowchart indicating a reprocessing method for systems, treatment instruments and components disclosed in the present application.

### DETAILED DESCRIPTION

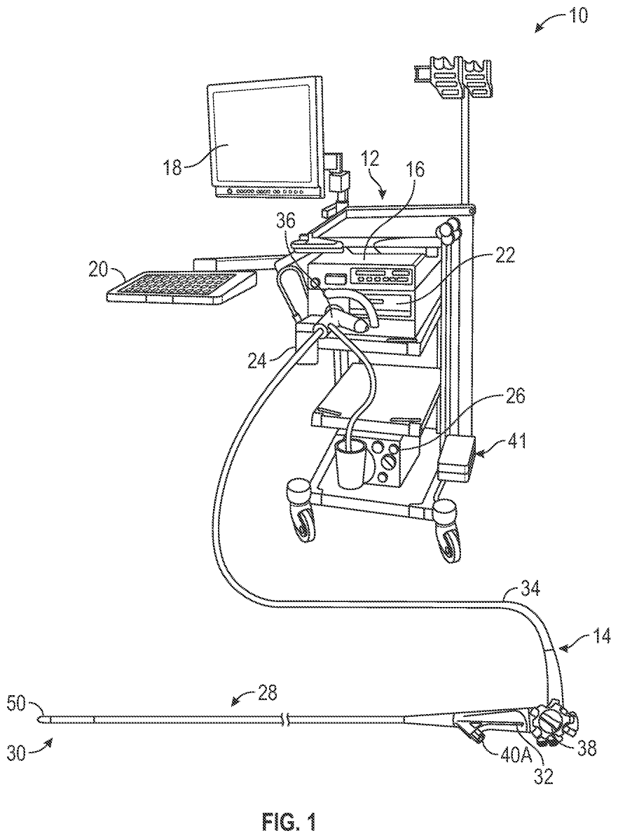

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as tensioning mechanisms for pull wires. However, the pull wire devices and methods of the present disclosure can be used in other configurations of endoscopy systems as well. According to some examples, endoscope 14 can be insertable into an anatomical region for imaging and/or to provide passage of other devices, such as auxiliary scopes and biopsy devices or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by a user, e.g., an operator, of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36. Coupler section 36 can be connected to control unit 16 to connect to endoscope 14 to multiple features of control unit 16, such as input unit 20, light source unit 22, fluid source 24 and suction pump 26.

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). In examples, a pair of pull wires can be anchored at functional section 30, extend through insertion section 28 and can be connected to control knob 38 to control bending or deflecting of the bending section. Insertion section 28 can also include one or more working channels (e.g., an internal lumen) that can be elongate and support insertion of one or more therapeutic tools of functional section 30, such as an auxiliary scope. The working channel can extend between handle section 32 and functional section 30. Additional functionalities, such as fluid passages, guide wires, and pull wires can also be provided by insertion section 28 (e.g., via suction or irrigation passageways, and the like).

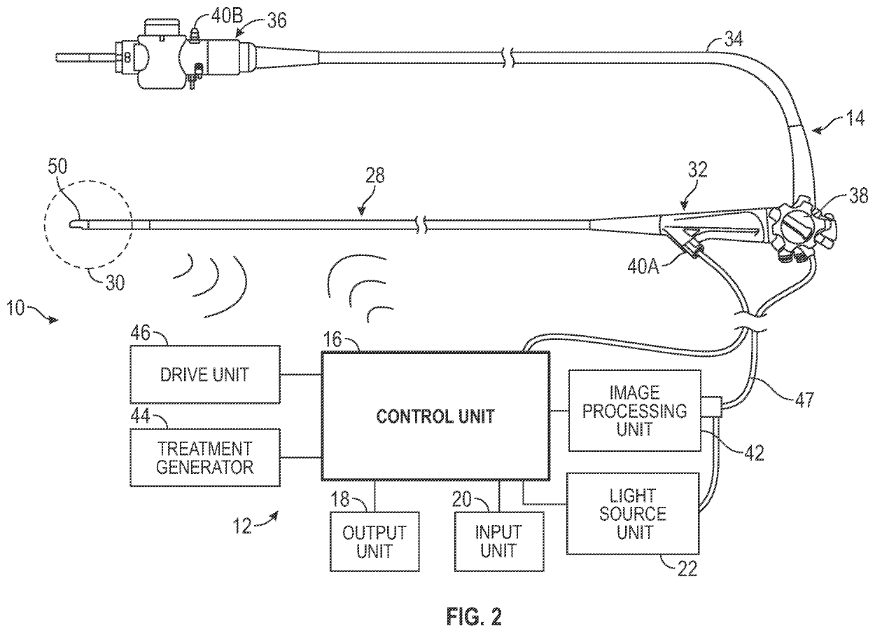

Handle section 32 can comprise control knob 38 as well as port 40A. As mentioned, control knob 38 can be coupled to a pull wire, or other actuation mechanisms, extending through insertion section 28. In examples, handle section 32 can include levers, wheels or other control elements for pushing and pulling of pull wires. Port 40A, as well as other ports, such as port 40B (FIG. 2), can be configured to couple various electrical cables, guide wires, auxiliary scopes, tissue collection devices, fluid tubes and the like to handle section 32 for coupling with insertion section 28.

Imaging and control system 12, according to examples, can be provided on a mobile platform (e.g., cart 41) with shelves for housing light source unit 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

Functional section 30 can comprise components for treating and diagnosing anatomy of a patient. Functional section 30 can comprise module 50 comprising an imaging device, an illumination device and an elevator.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source unit 22, input unit 20 and output unit 18. Coupler section 36 can be connected to control unit 16, as shown in FIG. 1, to connect to endoscope 14 to multiple features of control unit 16, such as image processing unit 42 and treatment generator 44. In examples, port 40A can be used to insert another instrument or device, such as a daughter scope or auxiliary scope, into endoscope 14. Such instruments and devices can be independently connected to control unit 16 via cable 47. In examples, port 40B can be used to connect coupler section 36 to various inputs and outputs, such as video, air, light and electric. Control unit 16 can be configured to activate a camera to view target tissue distal of endoscope 14. Likewise, control unit 16 can be configured to activate light source unit 22 to shine light on endoscope 14 or other devices extending therefrom.

Image processing unit 42 and light source unit 22 can each interface with endoscope 14 (e.g., at functional section 30) by wired or wireless electrical connections. Imaging and control system 12 can accordingly illuminate an anatomical region, collect signals representing the anatomical region, process signals representing the anatomical region, and display images representing the anatomical region on output unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

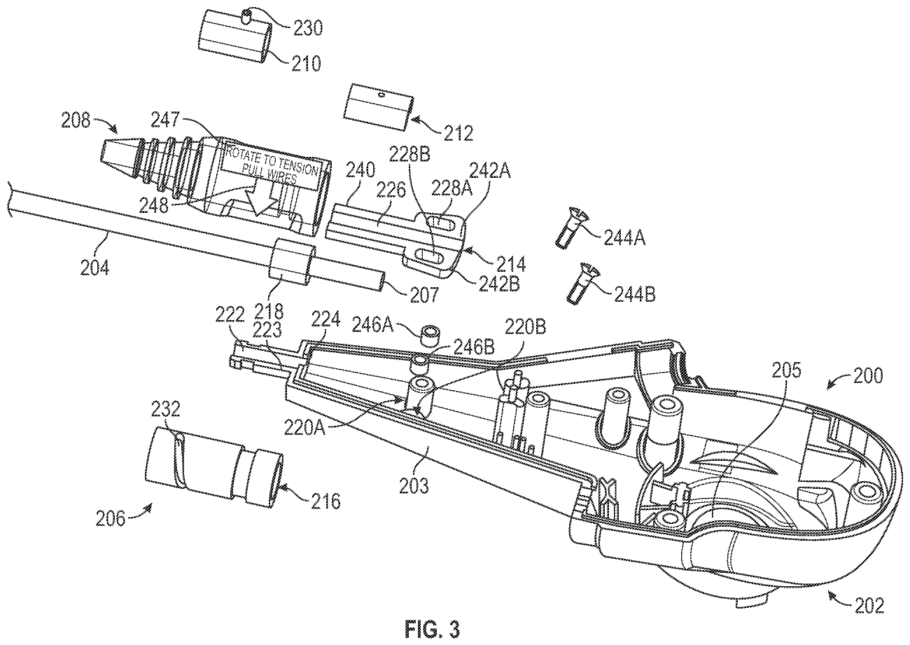

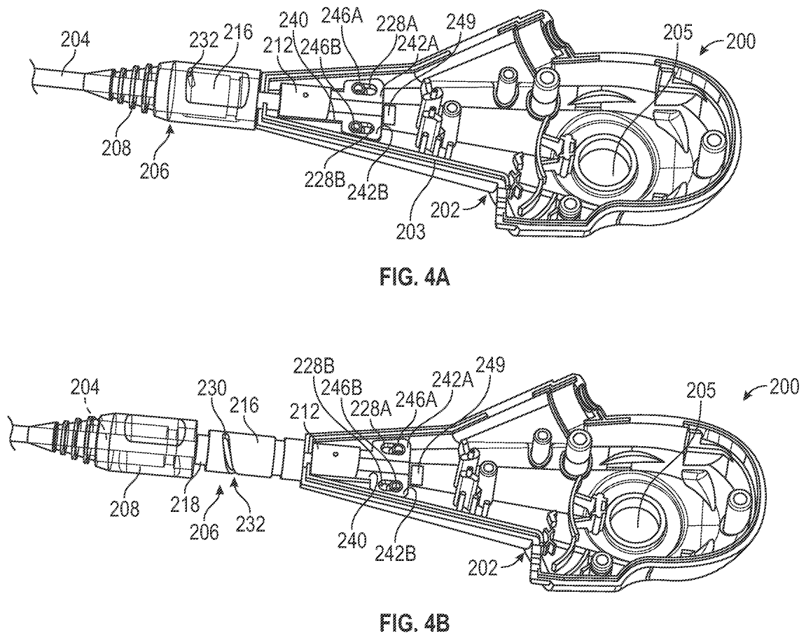

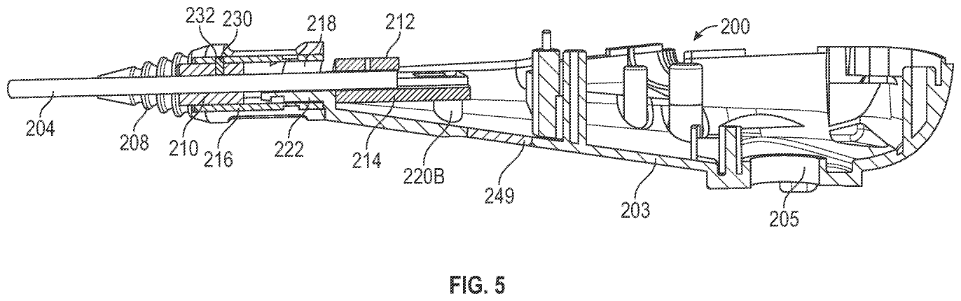

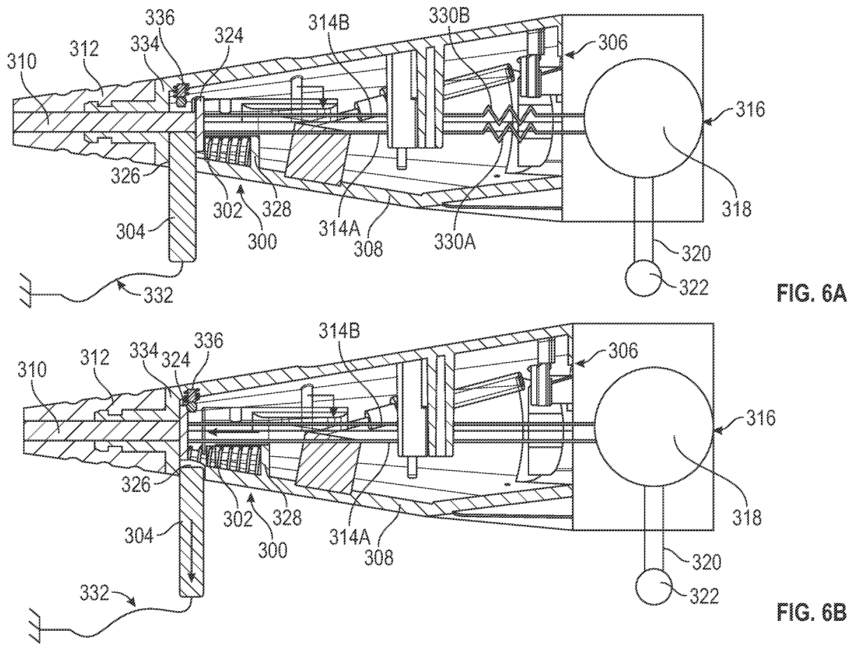

FIG. 3 is an exploded view of endoscope controller 200 comprising handle section 202, shaft 204 and tensioning mechanism 206, which can comprise a first example of a tensioning mechanism according to the present disclosure. Tensioning mechanism 206 can comprise strain relief 208, sheath collar 210, cap 212, shaft hub 214, rotary mechanism 216, stop 218 and standoffs 220A and 220B. FIG. 4A is a side view of tensioning mechanism 206 of FIG. 3 with shaft 204 in a retracted state. FIG. 4B is a side view of tensioning mechanism 206 of FIG. 3 with shaft 204 in an extended state. Strain relief 208 is displaced distally in FIG. 4B to show sheath collar 210 and rotary mechanism 216. FIG. 5 is top view of tensioning mechanism 206 of FIGS. 3-4B showing geometry of rotary mechanism 216. FIGS. 3-5 are discussed concurrently.

Handle section 202 can comprise controller housing 203, only half of which is shown in FIG. 3. Controller housing 203 can be split into two shell portions that are mirror images of each other. The two halves of controller housing 203 can be joined together to enclose tensioning mechanism 206. Controller housing 203 can include socket 205 for receiving a pull wire actuator, such as a lever or knob.

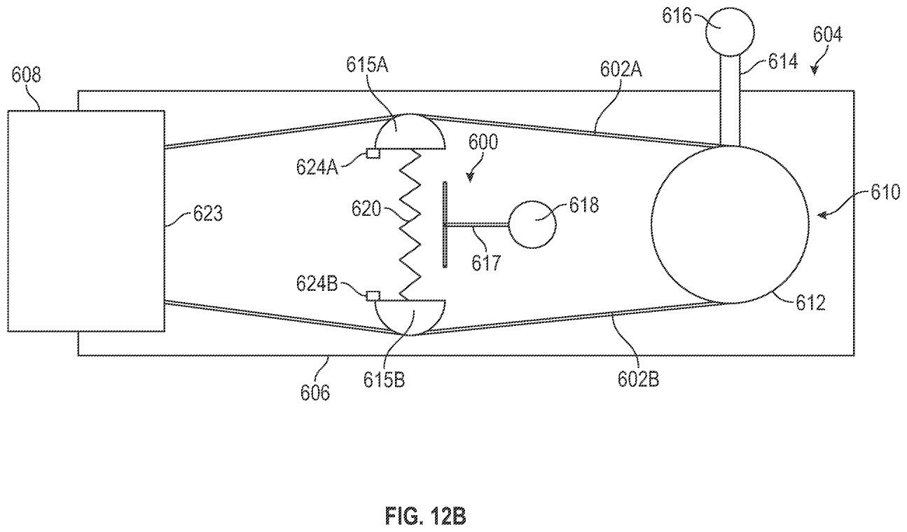

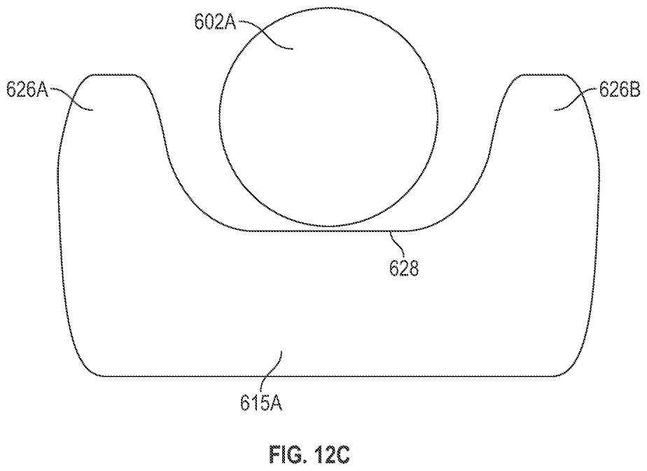

Shaft 204 can lay in socket 222 in handle section 202 and can be configured to slide freely therein. Shaft 204 can include pull wires extending from a proximal end and that can be connected to a drum or barrel positioned at socket 205. For example, shaft 204 and controller housing 203 can be configured as shaft 608 and handle housing 606 of FIGS. 12A and 12B, and an actuator such as actuator 610 can be positioned in socket 205. As such, handle section 202 can include pull wires similar to pull wire 602A and pull wire 602B shown in FIGS. 12A and 12B.

As discussed herein, handle section 202 can be subject to manufacturing and environmental conditions that can introduce slack into the pull wires, which ultimately can produce knob dwell in the actuator for the pull wires. As such, the pull wire can include slack. Tensioning mechanism 206 can be configured to eliminate or reduce the slack from the pull wires.

The pull wires can be fixed at the distal tip of shaft 204. The pull wires can be sized, e.g., have a length, to accommodate a particular distance between socket 205 and the distal tip of shaft 204, accommodating any fixed obstacles with controller housing 203. Thus, as shaft 204 changes shape, e.g., becomes reduced in length, proximal end face 207 of shaft 204 can become closer to socket 205 as compared to before shaft 204 changed shape, resulting in the generation of slack. In examples, shaft 204 can shrink, thereby shortening in length so that the pull wires are longer than desirable to produce a change in the position of the distal tip of shaft 204 when the actuator is displaced, e.g., longer than desirable to produce no or very little knob dwell. In short, the pull wires can be longer than needed to extend between socket 205 and the distal tip of shaft 204.

Stop 218 can be positioned around and fixed to shaft 204 opposite to tip 223. Sheath collar 210 can be positioned around shaft 204 forward of stop 218 and tip 223. Rotary mechanism 216 can be positioned around sheath collar 210 so that pin 230 extends into channel 232. Strain relief 208 can be positioned around rotary mechanism 216. Strain relief 208 can be positioned around shaft 204 to prevent kinking or sharp bending of shaft 204 at the harder material of tip 223 of controller housing 203, thereby preventing the generation of strain within the material of shaft 204. Sheath collar 210, rotary mechanism 216 and strain relief 208 can be configured to move relative to controller housing 203 via coupling to shaft 204. That is, as shaft 204 moves or slides within socket 222, the components of sheath collar 210, rotary mechanism 216 and strain relief 208 can move accordingly, either in a translational motion or a rotational motion, as explained below.

Shaft 204 can be attached to controller housing 203 via shaft hub 214. Shaft hub 214 can comprise slider 240 and lobes 242A and 242B. Proximal end of shaft 204 can be attached to channel 226 of shaft hub 214 to prevent movement of shaft 204. Cap 212 can be positioned over shaft 204 opposite shaft hub 214. Cap 212 and shaft hub 214 can be attached to shaft 204 by any suitable means, such as adhesive, bonding, fasteners and the like. Shaft hub 214 can be positioned so that slots 228A and 228B are positioned adjacent to standoffs 220A and 220B, respectively. Standoffs 220A and 220B can comprise pedestals against which shaft hub 214 can slide. Standoffs 220A and 220B can include bores for receiving fasteners 244A and 244B, respectively, that can pass through slots 228A and 228B. Heads of the fasteners 244A and 244B can hold lobes 242A and 242B against standoffs 220A and 220B, respectively. Bushings 246A and 246B can be positioned around the shafts of fasteners 244A and 244B, respectively, and positioned within slots 228A and 228B, respectively. Bushings 246A and 246B can facilitate alignment of shaft hub 214 with socket 222 and smooth sliding or translation of shaft hub 214.

As can be seen in FIG. 5, strain relief 208 can comprise a cylindrical body having an internal lumen in which rotary mechanism 216 can be placed. The outer surface of rotary mechanism 216 can fit tightly within the lumen of strain relief 208 so that strain relief 208 and rotary mechanism 216 move, e.g., rotate, together. Rotary mechanism 216 can comprise a cylindrical body having an internal lumen into which sheath collar 210 can be placed. The outer surface of sheath collar 210 can fit loosely within the lumen of rotary mechanism 216 so that rotary mechanism 216 can rotate about sheath collar 210. Sheath collar 210 can comprise a cylindrical body having an internal lumen into which shaft 204 can be placed. The outer surface of shaft 204 can fit tightly within the lumen of sheath collar 210 so that shaft 204 and sheath collar 210 move, e.g., translate, together. As discussed below, interaction of sheath collar 210 and rotary mechanism 216 can convert rotational movement of strain relief 208 to axial movement of shaft 204.

Rotation of strain relief 208 can cause rotary mechanism 216 to rotate, which can cause channel 232 of rotary mechanism 216 to push pin 230 and thereby push sheath collar 210. The axial position of strain relief 208 relative to controller housing 203 can be fixed, such as by the use of a flange on strain relief 208 or rotary mechanism 216 being positioned in a channel within controller housing 203 or via a similar mechanism. Shaft 204 can be constrained from rotating by coupling to shaft hub 214, thereby also preventing sheath collar 210 from rotating. The curved, helical or spiral shape of channel 232 can convert rotational movement of rotary mechanism 216 to axial movement of sheath collar 210 via sliding of pin 230 within channel 232. Because sheath collar 210 can be attached to shaft 204, movement of sheath collar 210 can cause shaft 204 to move. Movement of shaft 204 can adjust the tension or slack in pull wires located in shaft 204. Tensioning mechanism 206 can comprise a helical-drive tensioning mechanism.

In examples, strain relief 208 can be provided with markings, indicia or instructions indicating which way or how far to rotate strain relief 208 to reduce or eliminate pull wire slack and knob dwell. In the illustrated example, strain relief 208 can be configured to be rotated downward with respect to the orientation of FIG. 3 to cause tightening of the pull wires. Downward rotation of strain relief 208 can cause leftward movement of shaft 204. Strain relief 208 can thereby be provided with indicia, such as written alpha-numeric text 247 and icons, such as arrow 248, indicating the proper direction to rotate strain relief 208 to provide pull wire tensioning.

As shown in FIG. 4A, shaft hub 214 can be located proximally, or to the right in FIG. 4A after manufacturing. As such, shaft hub 214 can be spaced from end wall 224 and bushing 246A and bushing 246B can be at the proximal ends of slot 228A and slot 228B, respectively. Shrinkage of shaft 204 can cause the distal tip of shaft 204 to move proximally, thereby introducing slack into the pull wires. Strain relief 208 can be rotated one way, e.g., clockwise when looking proximally along shaft 204 from the distal end, to position shaft hub 214 distally as shown in FIG. 4B, thereby applying tension to the pull wires and removing slack therefrom. As such, shaft hub 214 can be engaged with end wall 224 and bushing 246A and bushing 246B can be located at the distal ends of slot 228A and slot 228B, respectively.

In examples, rotation of strain relief 208 can be a binary action, e.g., an on/off movement, so that a user of endoscope controller 200 does not need to determine how far to rotate strain relief 208. As such, strain relief 208 can be provided with stops for the extreme end positions of rotation. In examples, the stops can simply comprise one or both of end wall 224 or the ends of slot 228A and slot 228B. In examples, the stops can comprise detents, e.g., spring-loaded balls, that can become seated in a groove to lock relative movement, that can allow strain relief 208 to be snapped into place at the rotation ends. In examples, there could be intermediate steps between the rotation ends to allow a user to select the amount of tensioning to apply to the pull wires. For example, strain relief 208 could be provided with intermediate detents.

In examples, controller housing 203 can include window 249 to show the proximal end of shaft 204 or shaft hub 214. In examples, window 249 can be provided with indicia, such as a red/green gauge to provide user with an indication of a tension level in the pull wires to determine if tightening is needed or desired. For example, if the proximal end of shaft hub 214 is positioned over a red indicator, it can indicate that additional tightening, e.g., rotation of strain relief 208 is advantageous, or if the proximal end of shaft hub 214 is positioned over a green indicator, it can indicate that the tightening of the pull wires and the position of strain relief 208 is acceptable. Such a gauge, or another go/no-go gauge positioned on controller housing 203 or shaft hub 214 or somewhere else, can provide useful feedback to a user where strain relief 208 is not configured in a “set it and forget it” or “on/off” configuration. Additionally, such a gauge can be useful for a user to select a desired amount of knob dwell.

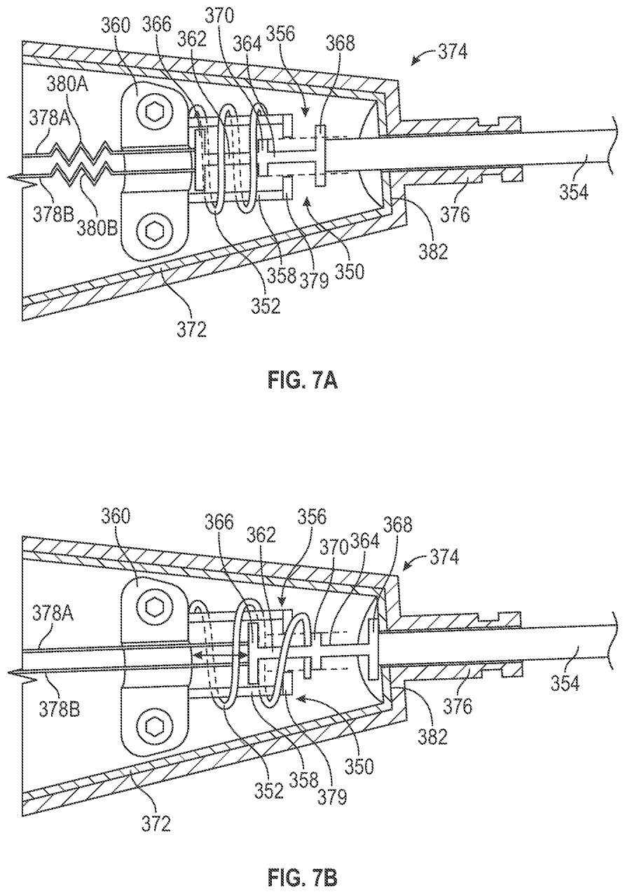

FIGS. 6A and 6B are side views of spring-loaded tensioning mechanism 300 comprising spring 302 and pin 304. Spring-loaded tensioning mechanism 300 can be incorporated into endoscope controller 306 comprising handle housing 308, to which shaft 310 is connected. Shaft 310 can be provided with strain relief 312. Handle housing 308 and shaft 310 can be configured similarly as described with reference to FIGS. 3-5. Strain relief 312 can be provided around shaft 310 to prevent strain from being induced in shaft 310 from bending at handle housing 308.

Pull wires 314A and 314B can extend from shaft 310 to actuator 316. Actuator 316 can comprise wheel 318, lever 320 and knob 322. In examples, pull wires 314A and 314B can comprise opposite end portions of a single wire wrapped around wheel 318. In examples, pull wires 314A and 314B can comprise two different wires connected to wheel 318.

Spring-loaded tensioning mechanism 300 can be disposed within or on handle housing 308 to interact with pull wires 314A and 314B. Spring-loaded tensioning mechanism 300 can comprise spring 302, flange 324 of shaft 310 and pin 304. Handle housing 308 can include opening 326 to receive pin 304 and flange 328 to push against spring 302.

As discussed herein, endoscope controller 306 can be subject to manufacturing and environmental conditions that can introduce slack into pull wires 314A and 314B. As such, pull wires 314A and 314B can include slack 330A and 330B. Spring-loaded tensioning mechanism 300 can be configured to eliminate or reduce slack 330A and 330B from pull wires 314A and 314B, respectively.

Pull wires 314A and 314B can be fixed at the distal tip of shaft 310. Pull wires 314A and 314B can be sized, e.g., have a length, to accommodate a particular distance between actuator 316 and the distal tip of shaft 310, accommodating any fixed obstacles with handle housing 308. Thus, as shaft 310 changes shape, e.g., becomes reduced in length, flange 324 of shaft 310 can become closer to actuator 316 as compared to before shaft 310 changed shape, resulting in the generation of slack. In examples, shaft 310 can shrink, thereby shortening in length so that pull wires 314A and 314B are longer than desirable to produce a change in the position of the distal tip of shaft 310 when actuator 316 is operated, e.g., longer than desirable to produce no or very little knob dwell. In short, the pull wire 314A and pull wire 314B can be longer than needed to extend between actuator 316 and the distal tip of shaft 310.

Spring-loaded tensioning mechanism 300 can be configured to push or pull on pull wire 314A and pull wire 314B to increase the length that the pull wires must travel to reach the distal tip of shaft 310 from actuator 316 to remove slack 330A and 330B from pull wires 314A and 314B. Spring-loaded tensioning mechanism 300 can be configured so that spring 302 pushes shaft 310 when pin 304 is removed. In examples, a user can remove pin 304 upon removal of endoscope controller 306 from packaging, such as a tray. In examples, pin 304 can be connected to tether 332 that can be fixed to the packaging to facilitate removal.

As shown, in FIG. 6A, endoscope controller 306 can be disposed within packaging before use such that slack 330A and 330B are present in pull wires 314A and 314B, respectively. Shaft 310 can be retracted proximally after manufacturing and sterilization. In examples, pin 304 can be positioned between flange 324 and housing wall 334 when fully advanced within opening 326. Thus, spring 302 can be compressed between flange 324 of shaft 310 and flange 328 of handle housing 308. Tether 332 can include slack so that endoscope controller 306 can be removed from packaging.

As shown in FIG. 6B, pin 304 can be retracted within opening 326 or fully removed from handle housing 308 to allow shaft 310 to move distally. In particular, with pin 304 retracted, spring 302 can be freed to push against flange 324. The distance that shaft 310 can advance can be configured to compensate for the shrinkage of shaft 310 and the corresponding generation of slack 330A and 330B. Until a user moves pin 304, spring 302 does not apply tension or compression to shaft 310. Such a configuration can be advantageous in that spring 302 does not apply constant compression to shaft 310, which can cause additional deformation of shaft 310 under certain conditions. Tether 332 can become taught and break as a user removes endoscope controller 306 from packaging. Detent mechanism 336 can be included so that as shaft 310 is pushed distally, shaft 310 can lock into the distal position such that the force of spring 302 need not hold shaft 310 in place and shaft 310 cannot be retracted proximally, at least not without force beyond those that are typically generated in pull wire 314A and pull wire 314B. In examples, detent mechanism 336 can be configured to allow flange 324 to readily move distally past detent mechanism 336, but not proximally backward past detent mechanism. In examples, detent mechanism 336 can comprise a spring-loaded ball or wedge mounted to handle housing 308 that can roll or slide along shaft 310, and then move into a groove in shaft 310 to immobilize axial movement of shaft 310.

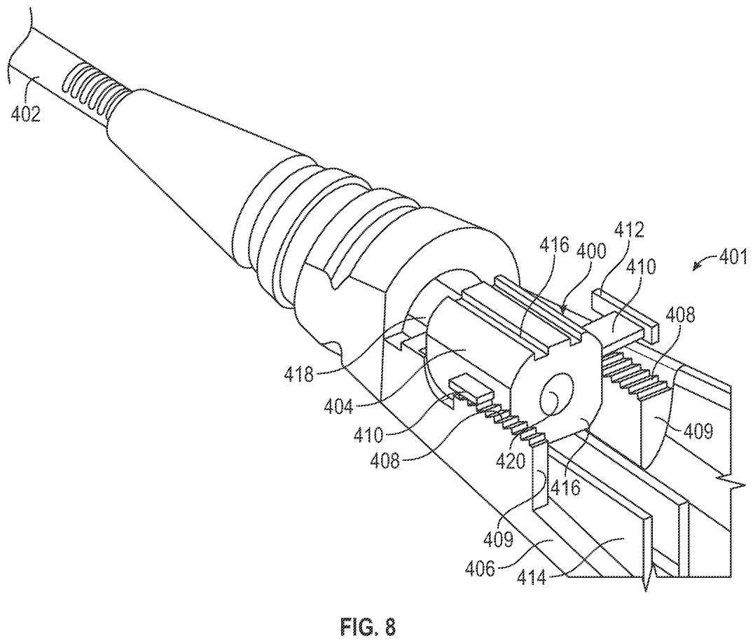

FIGS. 7A and 7B are side views of spring-loaded tensioning mechanism 350 in retracted and advanced states, respectively. Spring-loaded tensioning mechanism 350 can comprise spring 352 for pushing against shaft 354. Spring-loaded tensioning mechanism 350 can further comprise shuttle 356, guide body 358 and backstop 360. Shuttle 356 can comprise proximal portion 362, distal portion 364, proximal flange 366, distal flange 368 and middle flange 370. Spring-loaded tensioning mechanism 350 can be mounted within handle housing 372 of endoscope controller 374.

Endoscope controller 374 including handle housing 372 can be configured similarly as endoscope controller 200 and controller housing 203 of FIG. 3 and endoscope controller 306 and handle housing 308 of FIGS. 6A and 6B. Shaft 354 can be configured to slide within neck 376. Pull wire 378A and pull wire 378B can be configured to extend from shaft 354 to an actuation mechanism, such as an actuation mechanism similar to actuator 316 of FIGS. 6A and 6B.

In examples, spring 352 can be configured to continuously press against shaft 354 through shuttle 356. As shaft 354 shrinks, spring 352 can expand. Thus, spring 352 can be configured to continuously remove slack from pull wire 378A and pull wire 378B. However, in order to avoid inducing any additional stress within shaft 354, spring 352 can be configured to be selectively operated by a user. In additional examples, spring 352 can be initially set in a compressed state and held in place via a pin (not shown) similar to pin 304 of FIGS. 6A and 6B. The pin can be removed to push shaft 354 distally, such as before use, to remove slack.

The proximal end of shaft 354 can be connected to shuttle 356 having proximal flange 366 and distal flange 368 that engage flange 379 of guide body 358, thereby controlling the furthest extents of the movement of shaft 354. Spring 352 can push between backstop 360 and middle flange 370 to move shaft 354. In examples, proximal flange 366 and distal flange 368 can be taller than proximal portion 362 and distal portion 364 of shuttle 356 to engage with flange 379 of guide body 358, and middle flange 370 of shuttle 356 can be wider than proximal portion 362 and distal portion 364 of shuttle 356 to engage with spring 352. Middle flange 370 can be configured to fit between flanges 379 of shuttle 356. A detent mechanism (not shown) can be included so that as shaft 354 is pushed distally, shaft 354 can lock into the distal position such that the force of spring 352 need not hold shaft 354 in place and shaft 354 cannot be retracted proximally. In examples, the detent mechanism can comprise a spring-loaded ball or wedge mounted to handle housing 372 that can roll or slide along shaft 354 or shuttle 356, and then move into a groove in shaft 354 or shuttle 356 to immobilize axial movement of shaft 354.

As shown in FIG. 7A, shaft 354 can be positioned so that flange 379 of guide body 358 is between distal flange 368 sand middle flange 370 of shuttle 356. A pin (not shown) can hold shuttle 356 in place such that slack 380A and 380B can be present in pull wires 378A and 378B, respectively. As discussed herein, slack 380A and 380B can be induced by shrinkage of shaft 354 due to manufacturing and sterilization procedures or exposure to environmental heat.

As shown in FIG. 7B, a pin (not shown) can be removed from engagement with shuttle 356 and guide body 358 to allow shuttle 356 to freely move within guide body 358 under the force of spring 352. As such, spring 352 can push against middle flange 370 to advance shuttle 356 distally. In examples, shuttle 356 can be advanced distally until proximal flange 366 engages flange 379 of guide body 358 or distal flange 368 engages end wall 382 of handle housing 372. As such, slack 380A and 380B can be removed from pull wires 378A and 378B, respectively, due to pull wires 378A and 378B being attached to the distal end of shaft 354.

Furthermore, spring-loaded tensioning mechanism 350 can include a gauge to indicate the amount of tension being applied or indicia to provide instructions for operating spring-loaded tensioning mechanism 350.

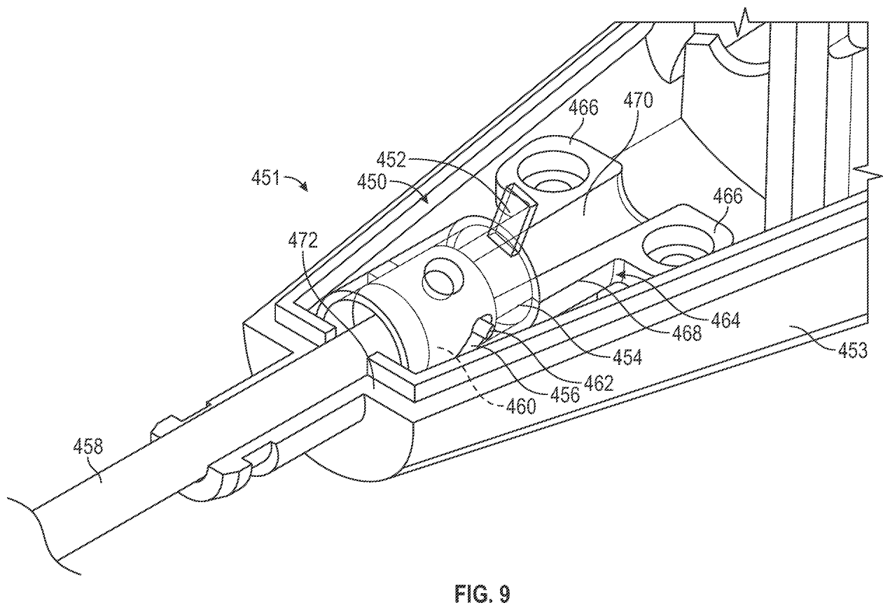

FIG. 8 is a perspective view of tensioning mechanism 400 comprising a ratchet mechanism incorporated into handle section 401. Shaft 402 can be attached to ratchet 404. Housing 406 can include teeth 408. Teeth 408 can be engaged by claws 410. A user can push lever 412 forward or distally to move shaft 402 forward. Teeth 408 and claws 410 can be configured to permit forward movement an prevent backward movement. Rails 414 of housing 406 can engage with slots 416 of ratchet 404 to maintain axial alignment of shaft 402 within housing 406.

Shaft 402 can lay in socket 418 in housing 406 and can be configured to slide freely therein. Shaft 402 can include pull wires extending from a proximal end and that can be connected to a drum or barrel positioned at a proximal portion of housing 406. For example, shaft 402 and housing 406 can be configured as shaft 608 and handle housing 606 of FIGS. 12A and 12B, and an actuator such as actuator 610 can be positioned in socket 205. As such, handle section 401 can include pull wires similar to pull wires 602A and 602B.

As discussed herein, handle section 401 can be subject to manufacturing and environmental conditions that can shrink shaft 402, which can introduce slack into the pull wires. Tensioning mechanism 400 can be configured to eliminate or reduce the slack from the pull wires.

The pull wires can be fixed at the distal tip of shaft 402. The pull wires can be sized, e.g., have a length, to accommodate a particular distance between a tensioner (not shown) and the distal tip of shaft 402, accommodating any fixed obstacles with housing 406. Thus, as shaft 402 changes shape, e.g., becomes shorter in length, proximal end face 420 of shaft 402 can become closer to the actuator as compared to before shaft 402 changed shape, resulting in the generation of slack. In examples, shaft 402 can shrink, thereby shortening in length so that the pull wires are longer than desirable to produce a change in the position of the distal tip of shaft 402 when the actuator is operated, e.g., longer than desirable to produce no or very little knob dwell. In short, the pull wires can be longer than needed to extend between the actuator and the distal tip of shaft 402.

In examples, handle section 401 can be removed from packaging for use. Ratchet 404 can be retracted proximally on rails 414. Ratchet 404 can be held in place on rails 414 by engagement of teeth of claws 410 with teeth 408 of ratchet strips 409 of housing 406. Springs can be incorporated to apply pressure to lever claws 410 to maintain engagement of the teeth. Lever 412 can extend through an opening in housing 406 to allow a user to interface with ratchet 404. As shaft 402 shrinks, the distal tip of shaft 402 can become closer to proximal end face 420 inducing slack in pull wires extending from proximal end face.

Before use of handle section 401, a user can push lever 412 distally or forward along housing 406 to move shaft 402 distally via ratchet 404 to remove the slack from the pull wires. Rails 414 can be inserted into slots 416 to facilitate sliding of ratchet 404. Rails 414 and slots 416 can be elongate to facilitate axial alignment of shaft 402 with housing 406. Furthermore, multiple rails 414 and slots 416 on opposite sides of ratchet 404 can prevent undesired rotation of ratchet 404. The teeth of claws 410 can be configured to engage with teeth 408 to allow forward or distal movement, but to prevent rearward or proximal movement. For example, rearward or proximal faces of teeth 408 can be inclined in the distal direction to allow claws 410 to advance distally, while forward or distal faces of teeth 408 can be vertical, e.g., perpendicular to the axis of rails 414, to prevent claws 410 from retreating proximally. Additionally, ratchet 404 can be provided with other features, such as detents, to prevent proximal migration of shaft 402. Furthermore, tensioning mechanism 400 can include a gauge to indicate the amount of tension being applied or indicia to provide instructions for operating tensioning mechanism 400.

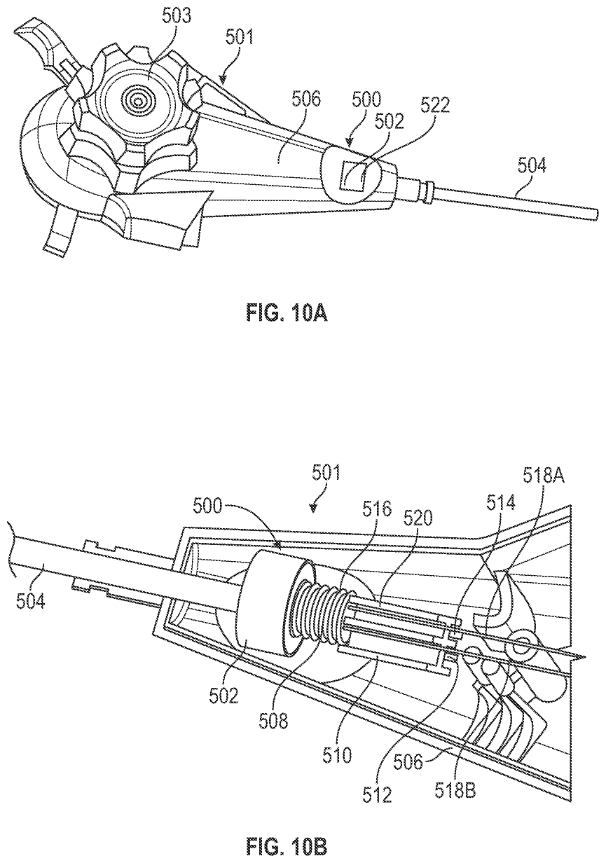

FIG. 9 is a perspective view of tensioning mechanism 450 comprising an internal screw mechanism incorporated into handle section 451. Tensioning mechanism 450 can comprise lever 452 that can be configured to extend outside of housing 453 allow a user to operate tensioning mechanism 450.

In examples, tensioning mechanism 450 can be configured similarly to tensioning mechanism 206 of FIGS. 3-5, but with the rotatable components being located within housing 453 and accessible via lever 452 rather than being operated by rotation of strain relief 208 (FIG. 3). Tensioning mechanism 450 can comprise a helical-drive tensioning mechanism.

Lever 452 can extend through a slot in housing 453 to couple to hub 454 having slot 456, which can be arcuate or spiral in shape. Shaft 458 can include collar 460 (shown in phantom within hub 454) having pin 462 configured to ride within slot 456. Hub 454 can be supported within housing 453 via connection to shaft hub 464. Shaft hub 464 can include wings 466 that can be secured to housing 453 with fasteners (not shown) and projection 468 that can extend into hub 454. Shaft hub 464 can additionally include channel 470 that can allow pull wires to access shaft 458. Hub 454 can be axially immobilized between a shoulder on projection 468 and wall 472 of housing 453.

Configured as such, hub 454 can be rotated by a user using lever 452. Rotational movement of hub 454 induced by lever 452 can cause slot 456 to impart forces to pin 462. The curvature of slot 456 can induce axial movement of collar 460 due to hub 454 being prevented from moving axially and collar 460 being allowed to rotate and move axially. Shaft 458 can be pushed forward by collar 460 and can be prevented from rotating by engagement of pin 462 with slot 456. FIG. 9 illustrates one of pin 462 and slot 456, but an additional pin and slot can be provided on the opposite side of collar 460 and hub 454.

In examples, tensioning mechanism 450 can include a spring assist to help push shaft 458 forward or distally. In examples, tensioning mechanism 450 can be provided with detents to hold lever 452 in advanced and retracted positions, as well as at intermediate positions to allow a user to set a desired amount of pull wire tension or knob dwell. Furthermore, tensioning mechanism 450 can include a gauge to indicate the amount of tension being applied or indicia to provide instructions for operating tensioning mechanism 450.

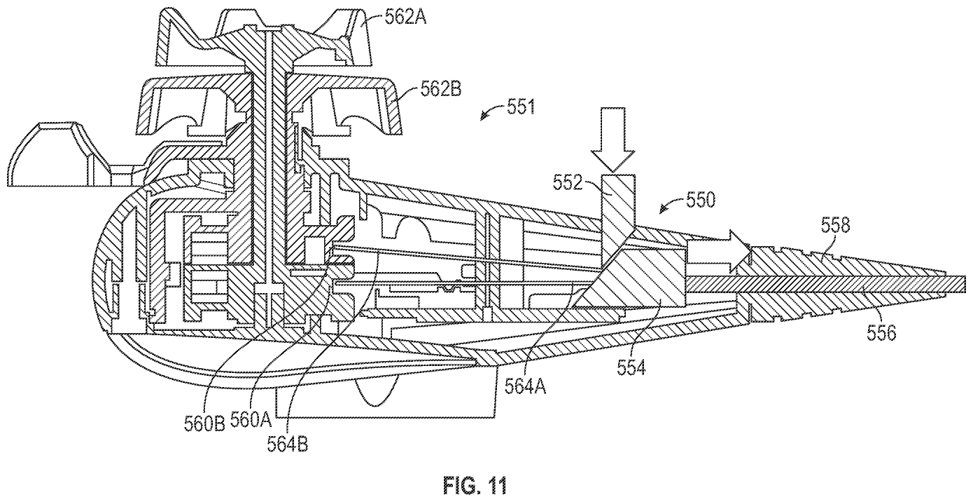

FIGS. 10A and 10B are external and internal views of screw-type tensioning mechanism 500 comprising an internal screw mechanism incorporated into handle section 501. Screw-type tensioning mechanism 500 can comprise knob 502 for moving shaft 504. Knob 502 can be accessible from an exterior of housing 506. Knob 503 can be connected to pull wires extending within shaft 504. Knob 503 can be rotated to push and pull the distal tip of shaft 504 using the pull wires. Knob 502 can be rotated to provide tension within the pull wires to eliminate or reduce knob dwell.

In examples, screw-type tensioning mechanism 500 can be configured similarly as tensioning mechanism 450 of FIG. 9, but with knob 502 replacing lever 452.

In examples, screw-type tensioning mechanism 500 can include a threaded engagement, such as a lead screw or jack screw to push and pull shaft 504 forward and backward. In such examples, screw-type tensioning mechanism 500 can comprise screw portion 508 and guide portion 510. Guide portion 510 can comprise a slide body having channels 512 configured to mate with ribs 514 extending from housing 506, similar to that of FIG. 8. Screw portion 508 can include bore 516 to allow pull wires 518A and 518B to pass through screw-type tensioning mechanism 500 and into shaft 504. Guide portion 510 can additionally include slot 520 to allow pull wires 518A and 518B to smoothly slide against and be guided out of screw-type tensioning mechanism 500.

Knob 502 can be rotated by an operator from the exterior of housing 506. In examples, knob 502 can be configured to rotate in only one direction. For example, knob 502 can be configured to only apply tension to pull wire 518A and pull wire 518B and not induce slack. Furthermore, knob 502 or housing 506 can be provided with indicia to indicate which way to rotate knob to tighten pull wires 518A and 518B. Knob 502 can be prevented from moving axially within housing 506 by positioning in window 522 of housing 506. Housing 506 can include a pair of windows, with one window on opposite sides of housing 506. Because knob 502 is prevented from moving axially, rotation of knob 502 can drive axial movement of screw portion 508 through threaded engagement. Knob 502 can be rotated to a plurality of positions between the proximal-most and distal-most positions of screw-type tensioning mechanism 500 to allow a user to control the amount of tension in pull wires 518A and 518B.

In examples, screw-type tensioning mechanism 500 can include a locking mechanism, such as a detent, to hold the shaft in a forward or distal position after knob 502 has been activated. In examples, the detent mechanism can comprise a spring-loaded ball or wedge mounted to housing 506 that can roll or slide along shaft 504 or guide portion 510, and then move into a groove in shaft 504 or guide portion 510 to immobilize axial movement of shaft 504.

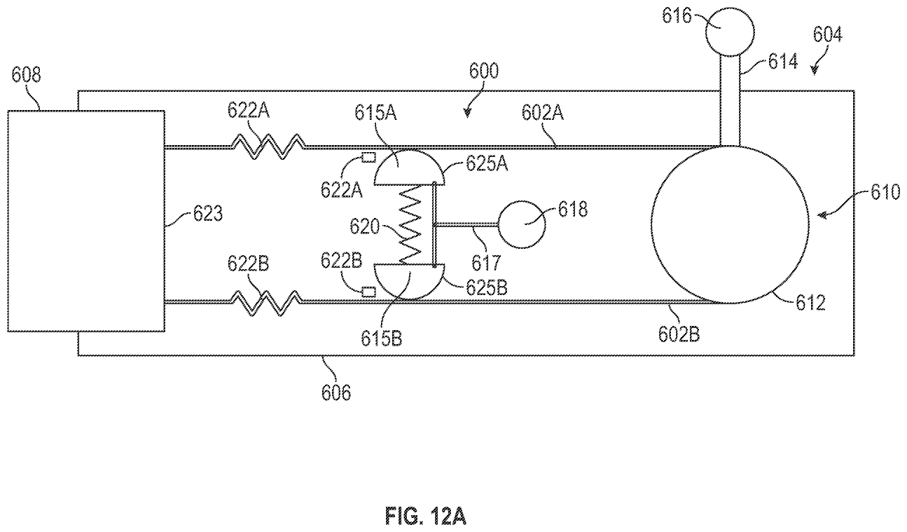

FIG. 11 is a schematic cross-sectional view of tensioning mechanism 550 comprising a button-activated wedge incorporated into handle section 551. Tensioning mechanism 550 can comprise button 552 and wedge 554. Wedge 554 can be located at the proximal end of shaft 556 or another location thereupon. Button 552 can extend through housing 558 to contact wedge 554. Button 552 and wedge 554 can have complimentary angled surfaces such that axial movement of button 552 into housing 558 can cause axial movement of wedge 554 out of housing 558. In examples, button 552 can move perpendicularly to wedge 554. In examples, tensioning mechanism 550 can include a locking mechanism, such as a detent, to hold shaft 556 in a forward or distal position or to hold button 552 or wedge 554 in a radially inward position after button 552 has been activated. In additional examples, a toggle linkage could be provided to lock button 552, wedge 554 or shaft 556 in position. The angle between button 552 and wedge 554 can be controlled, e.g., set, to determine, e.g., control, the amount of force applied to button 552 in order to displace shaft 556.

Housing 558 can comprise any housing described herein such as controller housing 203 of FIG. 3. Housing 558 can include a four-way pull wire system comprising first pulley 560A, second pulley 560B, first knob 562A and second knob 562B. First pulley 560A can be connected to pull wire pair 564A and second pulley 560B can be connected to pull wire pair 564B. Tensioning mechanism 550 can be configured so that wedge 554 simultaneously applies tension to all of the pull wires of pull wire pair 564A and pull wire pair 564B.

The examples of FIGS. 3-11 and FIGS. 16A and 16B can be configured to apply a translational, e.g., axial movement to an endoscope shaft to reduce or eliminate slack in one or more pull wires, such as by indirectly removing the slack through movement of the shaft. The various tensioning mechanisms can be configured to apply tension to a single pull wire, a pair of pull wires, or a pair of pull wire pairs. The examples of FIGS. 12A-15H can be configured to apply direct force to move, e.g., radially displace, one or more pull wires. In various examples of the present disclosure, a tensioning mechanism of FIGS. 3-11 and FIGS. 16A and 16B can be applied to a first pair of pull wires and a tensioning mechanism of FIGS. 12A-15H can be applied to a second pair of pull wires to allow for different tensioning of pull wire pairs. In examples, the different tensioning mechanisms of FIGS. 3-15H can be applied individually to one, two, three, four or more pull wires in various combinations to achieve individual pull wire tensioning.

FIG. 12A is a schematic cross-sectional view of spring-loaded tensioning mechanism 600 for pull wires 602A and 602B in a standby state. FIG. 12B is a schematic cross-sectional view of spring-loaded tensioning mechanism 600 of FIG. 12A in an activated state. FIGS. 12A and 12B are discussed concurrently.

Spring-loaded tensioning mechanism 600 can be used in endoscope controller 604. Endoscope controller 604 can comprise handle housing 606 and shaft 608. Handle housing 606 can be configured similarly as handle section 202 of FIG. 3 and shaft 608 can be configured similarly as shaft 204 of FIG. 3. Shaft 608 and handle housing 606 can be connected to each other in a fixed manner or can be connected to each other in a sliding manner as described with reference to FIGS. 3-5.

Pull wires 602A and 602B can extend from shaft 608 can be connected to actuator 610. Actuator 610 can comprise wheel 612, lever 614 and knob 616. In examples, pull wires 602A and 602B can comprise opposite end portions of a single wire wrapped around wheel 612. In examples, pull wires 602A and 602B can comprise two different wires connected to wheel 612.

Spring-loaded tensioning mechanism 600 can be disposed within or on handle housing 606 to interact with pull wires 602A and 602B. Spring-loaded tensioning mechanism 600 can comprise first tensioner 615A, second tensioner 615B, actuation mechanism 617, button 618, spring 620, first stop 624A and second stop 624B.

As discussed herein, endoscope controller 604 can be subject to manufacturing and environmental conditions that can introduce slack into pull wires 602A and 602B. As such, pull wires 602A and 602B can include slack 622A and 622B. Spring-loaded tensioning mechanism 600 can be configured to eliminate or reduce slack 622A and 622B from pull wires 602A and 602B, respectively.

Pull wires 602A and 602B can be fixed at the distal tip of shaft 608. Pull wires 602A and 602B can be sized, e.g., have a length, to accommodate a particular distance between actuator 610 and the distal tip of shaft 608, accommodating any fixed obstacles with handle housing 606. Thus, as shaft 608 changes shape, becomes reduced in length, proximal end face 623 of shaft 608 can become closer to actuator 610 as compared to before shaft 608 changed shape, resulting in the generation of slack. In examples, shaft 608 can shrink, thereby shortening in length so that pull wires 602A and 602B are longer than needed to adjust the distal tip of shaft 608, resulting in the generation of slack 622A and 622B. As shown in FIG. 12A, pull wires 602A and 602B can be longer than desirable to produce a change in the position of the distal tip of shaft 608 when the actuator 610 is operated, e.g., longer than desirable to produce no or very little knob dwell. In short, the pull wires 602A and 602B can be longer than needed to extend between actuator 610 and the distal tip of shaft 608.

Spring-loaded tensioning mechanism 600 can be configured to push or pull on pull wires 602A and 602B to increase the length that pull wires must travel to reach the distal tip of shaft 608 from actuator 610 to remove slack 622A and 622B from pull wires 602A and 602B. Spring-loaded tensioning mechanism 600 can be configured so that first tensioner 615A and second tensioner 615B are disengaged from pull wires 602A and 602B to not have an effect on the tension of pull wires 602A and 602B, as shown in FIG. 12A. However, first tensioner 615A and second tensioner 615B can be moved into engagement with pull wires 602A and 602B, respectively, to remove slack 622A and 622B, as shown in FIG. 12B. Tensioners 615A and 615B can increase the path length of pull wires 602A and 602B, respectively, by producing an indirect path between shaft 608 and actuator 610 to remove slack 622A and 622B. In the example of FIGS. 12A and 12B, tensioners 615A and 615B are illustrated as pushing pull wires 602A and 602B outward, but can be configured to pull or push pull wires 602A and 602B inward or outward.

First tensioner 615A and second tensioner 615B can comprise deflectors or other bodies that can be moved into engagement with pull wires 602A and 602B. In examples, tensioners 615A and 615B can be configured as stationary or rotatable pulleys. First tensioner 615A and second tensioner 615B can comprise arcuate surface 625A and arcuate surface 625B, respectively, against which pull wires 602A and 602B can slide. Thus, first tensioner 615A and second tensioner 615B can avoid inducing stress or kinks within pull wires 602A and 602B. In examples, as shown in FIG. 12C, arcuate surface 625A can comprise flanges 626A and 626B and trough 628 to maintain pull wire 602A aligned and engaged with first tensioner 615A. Second tensioner 615B can be configured similarly as first tensioner 615A.

Button 618 can be actuated to release tension in spring 620 to cause movement of first tensioner 615A and second tensioner 615B. Button 618 can be connected to actuation mechanism 617, which can comprise tethers or linkages connected to first tensioner 615A and second tensioner 615B. Depressing of button 618 can cause actuation mechanism 617 to release from first tensioner 615A and second tensioner 615B. In additional examples, a button can be provided individually for each of tensioners 615A and 615B.

In the illustrated example, first tensioner 615A and second tensioner 615B are configured to move outward from a location between pull wires 602A and 602B to push pull wires 602A and 602B outward. However, actuation mechanism 617 can be configured to pull on pull wires 602A and 602B from locations outside of pull wires 602A and 602B. In additional examples, tethers or linkages of actuation mechanism 617 can remain attached to first tensioner 615A and second tensioner 615B and actuation of button 618 can be configured to cause movement of first tensioner 615A and second tensioner 615B directly from the tethers or linkages. Thus, in examples, spring 620 can be omitted and a catch mechanism, e.g., one or more detents, can be included to maintain the tethers or linkages of actuation mechanism in the advanced or actuated state.

In the illustrated example, first tensioner 615A and second tensioner 615B and configured to act on pull wires 602A and 602B, respectively, at the same time. However, in additional examples, each of first tensioner 615A and second tensioner 615B can be provided with a dedicated actuation mechanism and button to allow for individual control over the tensioning of pull wires 602A and 602B.

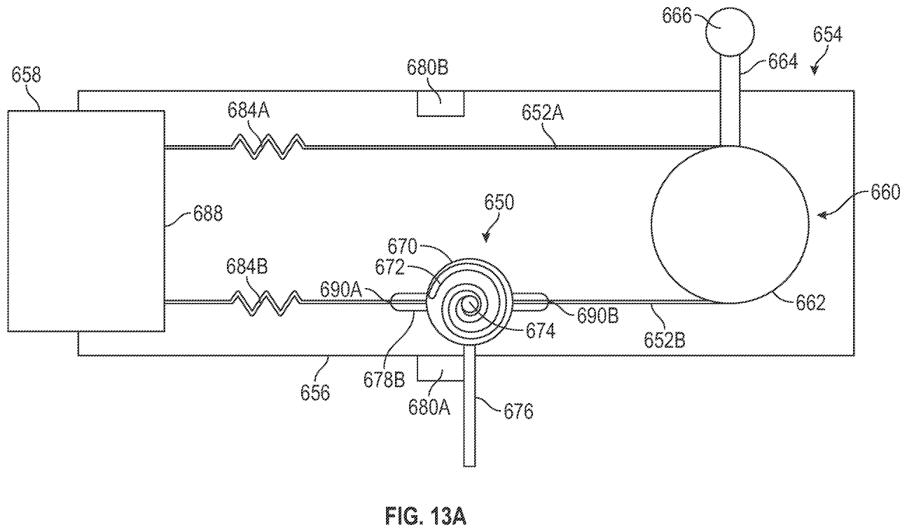

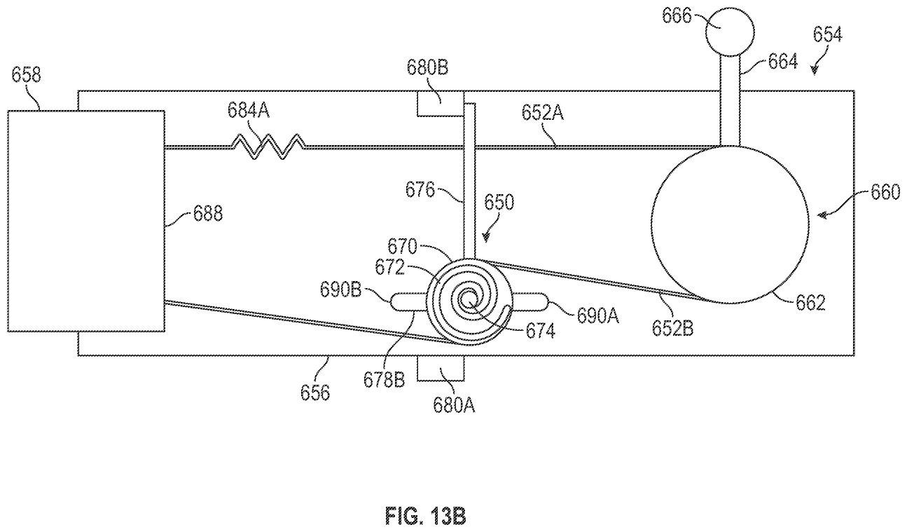

FIG. 13A is a schematic cross-sectional view of torsion spring tensioning mechanism 650 for pull wire 652A and pull wire 652B in a standby state. FIG. 13B is a schematic cross-sectional view of torsion spring tensioning mechanism 650 of FIG. 13A in an activated state. FIG. 13C is a schematic top view of torsion spring tensioning mechanism 650 of FIGS. 13A and 13B. FIGS. 13A-13C are discussed concurrently.

Torsion spring tensioning mechanism 650 can be used in endoscope controller 654. Endoscope controller 654 can comprise handle housing 656 and shaft 658. Handle housing 706 can be configured similarly as handle section 202 of FIG. 3 and shaft 658 can be configured similarly as shaft 204 of FIG. 3. Shaft 658 and handle housing 656 can be connected to each other in a fixed manner or can be connected to each other in a sliding manner as described with reference to FIGS. 3-5.

Pull wires 652A and 652B can extend from shaft 658 can be connected to actuator 660. Actuator 660 can comprise wheel 662, lever 664 and knob 666. In examples, pull wires 652A and 652B can comprise opposite end portions of a single wire wrapped around wheel 662. In examples, pull wires 652A and 652B can comprise two different wires connected to wheel 662.

Torsion spring tensioning mechanism 650 can be disposed within or on handle housing 656 to interact with pull wires 652A and 652B. Torsion spring tensioning mechanism 650 can comprise drum 670, torsion spring 672, spindle 674 and actuation mechanism 676. Spindle 674 can be configured to ride on slots 678A (FIG. 13C) and 678B. First stop 680A and second stop 680B can be mounted to handle housing 656 to engage with actuation mechanism 676. Actuation mechanism 676 can be connected to spindle 674 to rotate drum 670 between first stop 680A and second stop 680B.

As discussed herein, endoscope controller 654 can be subject to manufacturing and environmental conditions that can introduce slack into pull wires 652A and 652B. As such, pull wires 652A and 652B can include slack 684A and 684B. Torsion spring tensioning mechanism 650 can be configured to eliminate or reduce slack 684A and 684B from pull wires 652A and 652B, respectively.

Pull wires 652A and 652B can be fixed at the distal tip of shaft 658. Pull wires 652A and 652B can be sized, e.g., have a length, to accommodate a particular distance between actuator 660 and the distal tip of shaft 658, accommodating any fixed obstacles with handle housing 656. Thus, as shaft 658 changes shape, e.g., becomes reduced in length, proximal end face 688 of shaft 658 can become closer to actuator 660 as compared to before shaft 658 changed shape, resulting in the generation of slack. In examples, shaft 658 can shrink, thereby shortening in length so that pull wires 652A and 652B are longer than desirable to produce a change in the position of the distal tip of shaft 658 when actuator 660 is operated, e.g., longer than desirable to produce no or very little knob dwell. In short, pull wires 652A and 652B can be longer than needed to extend between actuator 660 and the distal tip of shaft 658.

Torsion spring tensioning mechanism 650 can be configured to wrap pull wire 652B around drum 670 to remove slack 684B from pull wire 652B. In a standby state, pull wire 652B can engaged with drum 670 so that pull wire 652A extends along a first path on or through drum 670, as shown in FIG. 13A. In an activated state, drum 670 can be rotated so that pull wire 652B engages with drum 670 so that pull wire 652A extends along a second path on or through drum 670 that is longer than the first path, as shown in FIG. 13B. Thus, pull wire 652B can be cinched within drum 670.

Drum 670 can be mounted on spindle 674, which can be supported by slot 678A and slot 678B, which can be formed in handle housing 656 or can comprise separate structure within handle housing 656. Slot 678A and slot 678B can be mounted to handle housing 656 in any suitable fashion. Actuation mechanism 676 can be connected to drum 670 to facilitate rotation of drum 670 on spindle 674. Actuation mechanism 676 can comprise a lever extending through handle housing 656 to allow for manipulation by a user. In examples, handle housing 656 can be provided with first stop 680A and second stop 680B to facilitate holding or immobilizing actuation mechanism 676 in the positions of FIGS. 13A and 13B, respectively. For example, first stop 680A and second stop 680B can comprise spring loaded detents. Torsion spring 672 can couple drum 670 to spindle 674 so that actuation mechanism 676 can be rotated differently than drum 670. That is, actuation mechanism 676 can rotate spindle 674 causing drum 670 to rotate via interaction with torsion spring 672. However, as slack 684B is removed from pull wire 652B, torsion spring 672 can be wound so that spindle 674 and actuation mechanism 676 can be further rotated to impart tension to pull wire 652B and to allow actuation mechanism 676 to engage second stop 680B.

In examples, drum 670 can comprise a cylindrical body. In examples, drum 670 can comprise a plate or paddle.

In examples, spindle 674 can be connected to slots 678A and 678B using ratchet mechanisms that allow one-way rotation of spindle 674.

As discussed herein, pull wire 652B can be attached to drum 670 to allow for winding of various lengths of pull wire 652B around drum 670 to remove slack 684B. As such, pull wire 652B is fixed to the location of drum 670. As such, drum 670 can be configured to be positioned within handle housing 656 in a moveable manner to allow for operation of actuator 660. For example, as actuator 660 rotates wheel 662 clockwise in FIGS. 13A and 13B, pull wire 652B will move to the left, and as actuator 660 rotates wheel 662 counter-clockwise in FIGS. 13A and 13B, pull wire 652B will move to the right. Thus, in order to avoid torsion spring tensioning mechanism 650 from anchoring pull wire 652B on handle housing 656 and inducing slack in pull wire 652B between torsion spring tensioning mechanism 650 and actuator 660 for clockwise rotation and tension for counter-clockwise rotation, drum 670 can move left and right within handle housing 656 on slots 678A and 678B. Thus, as actuator 660 rotates wheel 662 clockwise in FIGS. 13A and 13B, spindle 674 will move to the left on slots 678A and 678B toward distal end 690A, and as actuator 660 rotates wheel 662 counter-clockwise in FIGS. 13A and 13B, spindle 674 will move to the right on slots 678A and 678B toward proximal end 690B.

FIGS. 13A and 13B are illustrated as having torsion spring tensioning mechanism 650 configured to engage pull wire 652B. In additional examples, torsion spring tensioning mechanism 650 can be provided on pull wire 652A. In examples, torsion spring tensioning mechanism 650 can be provided on pull wire 652B and a similarly configured tensioning mechanism can be provided on pull wire 652A. In configurations using two rotatable tensioning mechanisms, the actuation mechanism, e.g., actuation mechanism 676, can be linked so that a single action can be used to simultaneously activate both actuation mechanisms.

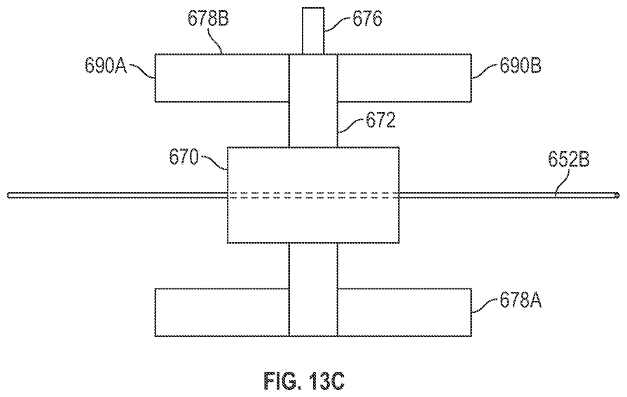

FIG. 14A is a schematic cross-sectional view of rotatable tensioning mechanism 700 for pull wires in a standby state. FIG. 14B is a schematic cross-sectional view of rotatable tensioning mechanism 700 of FIG. 14A in an activated state. FIGS. 14A and 14B are discussed concurrently.

Rotatable tensioning mechanism 700 can be used in endoscope controller 704. Endoscope controller 704 can comprise handle housing 706 and shaft 708. Handle housing 706 can be configured similarly as handle section 202 of FIG. 3 and shaft 708 can be configured similarly as shaft 204 of FIG. 3. Shaft 708 and handle housing 706 can be connected to each other in a fixed manner or can be connected to each other in a sliding manner as described with reference to FIGS. 3-5.

Pull wires 702A and 702B can extend from shaft 708 can be connected to actuator 710. Actuator 710 can comprise wheel 712, lever 714 and knob 716. In examples, pull wires 702A and 702B can comprise opposite end portions of a single wire wrapped around wheel 712. In examples, pull wires 602A and 602B can comprise two different wires connected to wheel 712.

Rotatable tensioning mechanism 700 can be disposed within or on handle housing 706 to interact with pull wires 702A and 702B. Rotatable tensioning mechanism 700 can comprise drum 717, tensioner 718, actuation mechanism 719, shaft 720, first bearing 723A and second bearing 723B.

As discussed herein, endoscope controller 704 can be subject to manufacturing and environmental conditions that can introduce slack into pull wires 702A and 702B. As such, pull wires 702A and 702B can include slack 722A and 722B. Rotatable tensioning mechanism 700 can be configured to eliminate or reduce slack 722A and 722B from pull wires 702A and 702B, respectively.

Pull wires 702A and 702B can be fixed at the distal tip of shaft 708. Pull wires 702A and 702B can be sized, e.g., have a length, to accommodate a particular distance between actuator 710 and the distal tip of shaft 708, accommodating any fixed obstacles with handle housing 706. Thus, as shaft 708 changes shape, e.g., becomes reduced in length, proximal end face 724 of shaft 708 can become closer to actuator 710 as compared to before shaft 708 changed shape, resulting in the generation of slack. In examples, shaft 708 can shrink, thereby shortening in length so that pull wires 702A and 702B are longer than desirable to produce a change in the position of the distal tip of shaft 708 when the actuator is displaced, e.g., longer than desirable to produce no or very little knob dwell. In short, pull wires 702A and 702B can be longer than needed to extend between actuator 710 and the distal tip of shaft 708.

Rotatable tensioning mechanism 700 can be configured to push or pull on pull wire 702B to increase the distance that pull wires must travel to reach the distal tip of shaft 708 from actuator 710 to remove slack 722B from pull wire 702B. Rotatable tensioning mechanism 700 can be configured so that drum 717 is rotated so that tensioner 718 is disengaged from pull wire 702B to not have an effect on the tension of pull wire 702B, as shown in FIG. 14A. However, drum 717 can be rotated to move tensioner 718 into engagement with pull wire 702B to remove slack 722B, as shown in FIG. 14B.

Drum 717 can be mounted on shaft 720, which can be supported by first bearing 723A and second bearing 723B. In examples, shaft 720 can extend parallel, or nearly parallel, to the direction of axis of pull wire 702B. First bearing 723A and second bearing 723B can be mounted to handle housing 706 in any suitable fashion. First bearing 723A and second bearing 723B can facilitate rotation of shaft 720. Actuation mechanism 719 can be connected to drum 717 to facilitate rotation of drum 717 on shaft 720. Actuation mechanism 719 can comprise a lever extending through handle housing 706 to allow for manipulation by a user. In examples, handle housing 706 can be provided with latch 726A and latch 726B to facilitate holding or immobilizing actuation mechanisms 719 in the positions of FIGS. 14A and 14B, respectively. For example, latches 726A and 726B can comprise spring loaded detents. In examples, a spring, such as a coil spring can be connected to drum 717 to facilitate advancement of drum 717 to the actuated position of FIG. 14B once actuation mechanism 719 is freed from latch 726A.

In examples, drum 717 can comprise a cylindrical body. In examples, drum 717 can comprise a plate or paddle.

In examples, first bearing 723A and second bearing 723B can include or can comprise ratchet mechanisms that allow one-way rotation of shaft 720.

Tensioner 718 can comprise a post or peg that can push on pull wire 702B. Tensioner 718 can have a rounded or curved shape to prevent kinking or binding of pull wire 702B. In examples, tensioner 718 can include a hole or through bore in the post or peg. Pull wire 702B can extend through the hole or through bore to ensure that pull wire 702B does not slip off of or otherwise separate from tensioner 718. In examples, tensioner 718 can comprise a hoop or wicket attached to drum 717 and through which pull wire 702B can extend. In examples, handle housing 706 can include additional guides, such as bumpers or rails, to guide pull wire 702B from shaft 708, around drum 717 and to actuator 710.

FIGS. 14A and 14B are illustrated as having rotatable tensioning mechanism 700 configured to engage pull wire 702B. In additional examples, rotatable tensioning mechanism 700 can be provided on pull wire 702A. In examples, rotatable tensioning mechanism 700 can be provided on pull wire 702B and a similarly configured tensioning mechanism can be provided on pull wire 702A. In configurations using two rotatable tensioning mechanisms, the actuation mechanism, e.g., actuation mechanism 719, can be linked so that a single action can be used to simultaneously activate both actuation mechanisms. In additional examples, drum 717 can be sized so that opposite sides of drum 717 are positioned proximate pull wires 702A and 702B and the opposite sides of drum 717 can be provided with a tensioner, e.g., tensioner 718, to engage pull wires 702A and 702B. Thus, a single drum and a single actuation movement can be used to tension both of pull wires 702A and 702B simultaneously.

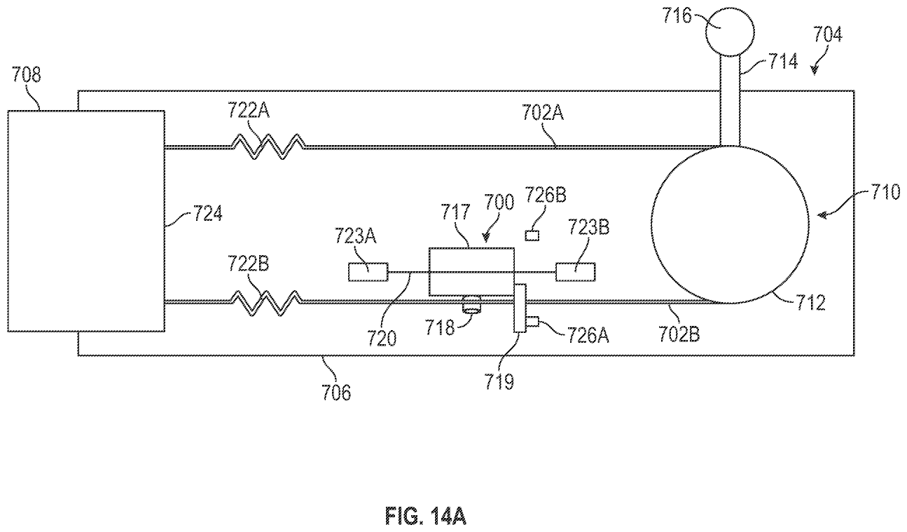

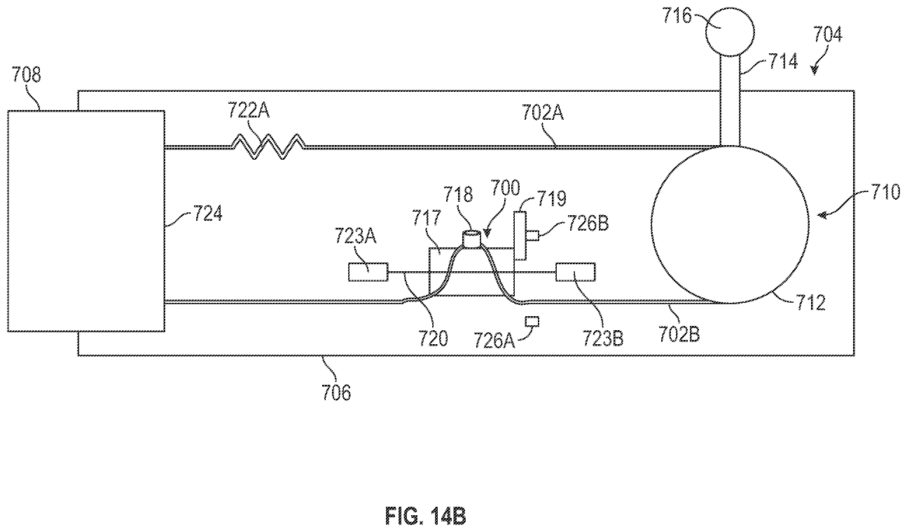

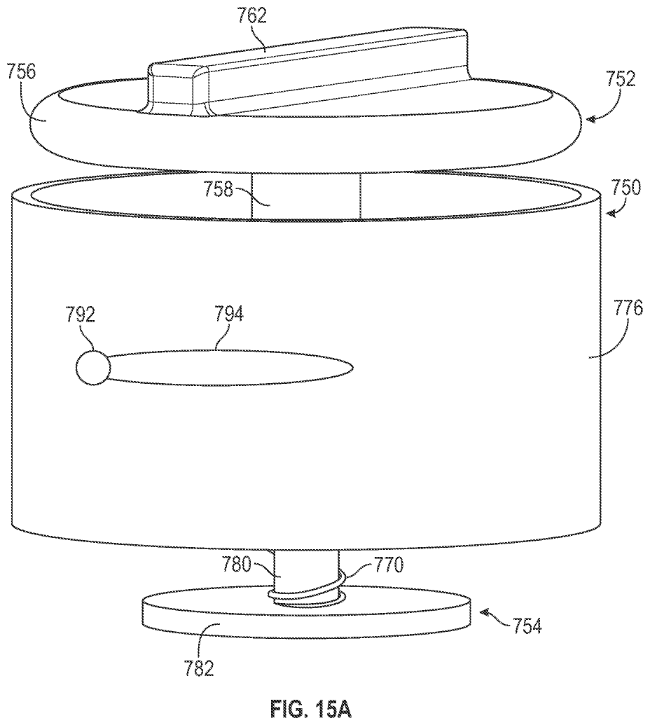

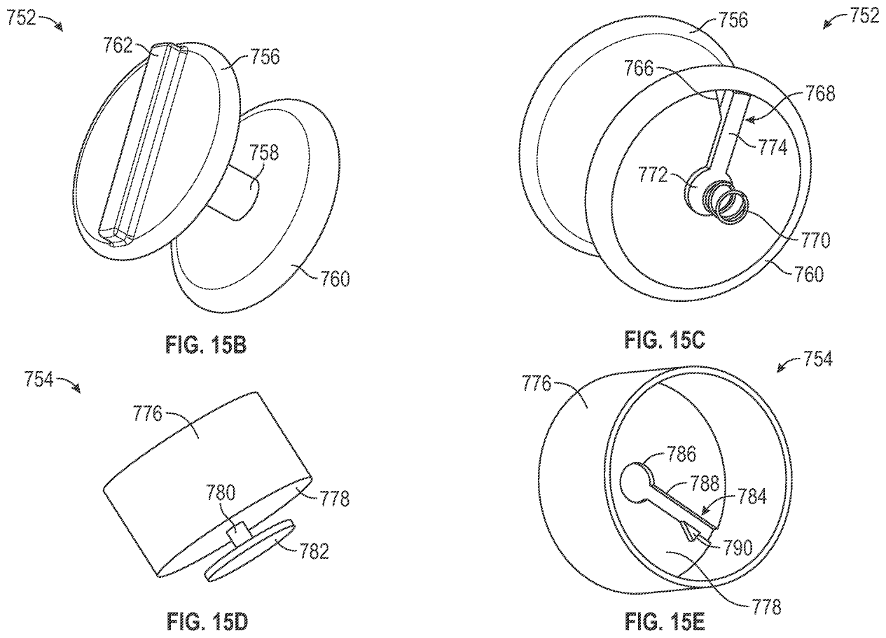

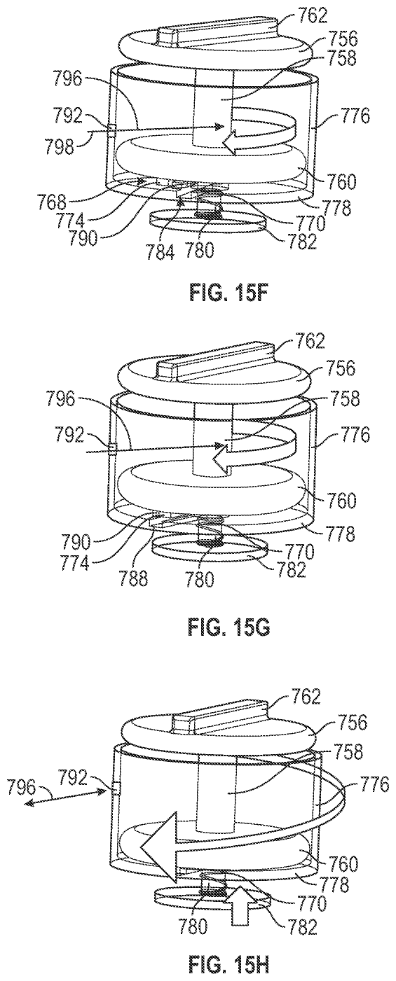

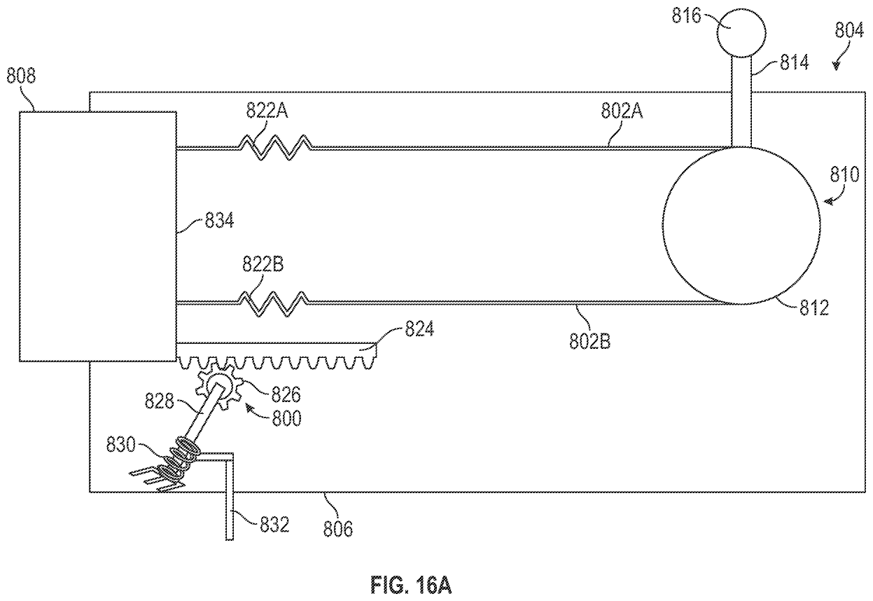

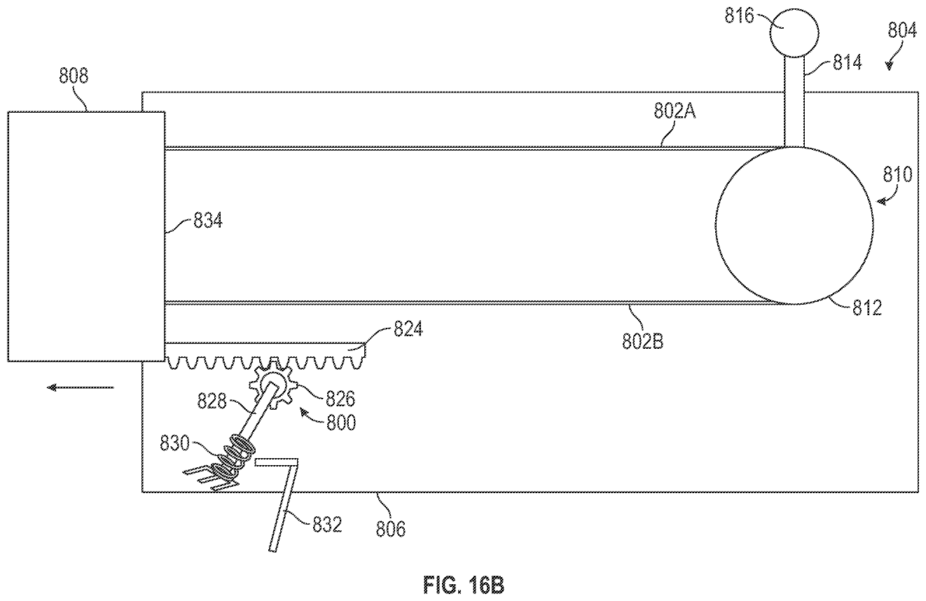

FIG. 15A is a perspective view of rotatable barrel tensioning mechanism 750 of the present disclosure comprising knob component 752 and barrel component 754. Rotatable barrel tensioning mechanism 750 can be incorporated into an actuator for operating pull wires of an endoscope controller. In examples, rotatable barrel tensioning mechanism 750 can be incorporated into actuator 610 of FIG. 12A, actuator 660 of FIG. 13A, actuator 710 of FIG. 14A or actuator 810 of FIG. 16A, as well as other actuator suitable for use with the systems of FIGS. 3-11. Rotatable barrel tensioning mechanism 750 can be used as an alternative to spring-loaded tensioning mechanism 600 of FIG. 12A, torsion spring tensioning mechanism 650 of FIG. 13A, rotatable tensioning mechanism 700 of FIG. 14A and rack and pinion tensioning mechanism 800 of FIG. 16A. However, in various examples, rotatable barrel tensioning mechanism 750 can be used in conjunction with spring-loaded tensioning mechanism 600 of FIG. 12A, torsion spring tensioning mechanism 650 of FIG. 13A, rotatable tensioning mechanism 700 of FIG. 14A and rack and pinion tensioning mechanism 800 of FIG. 16A or other mechanisms of FIGS. 3-11.

FIG. 15B is a perspective view of a first side of knob component 752 of FIG. 15A. FIG. 15C is a perspective view of a second side of knob component 752 of FIG. 15A. Knob component 752 can comprise knob disk 756, knob shaft 758 and clutch disk 760. Knob disk 756 can comprise knob 762. As shown in FIG. 15C, clutch disk 760 can comprise stop slot 766, and knob component 752 can further comprise clutch 768 and spring 770. Clutch 768 can comprise pivot 772 and extension 774.

FIG. 15D is a perspective view of a first side of barrel component 754 of FIG. 15A. FIG. 15E is a perspective view of a second side of barrel component 754 of FIG. 15A. Barrel component 754 can comprise barrel end wall 776, barrel side wall 778, post 780 and base 782. Barrel side wall 778 can comprise clutch socket 784, which can include center portion 786 and extension portion 788, and stop 790.

In operation, pull wires can be attached to or extended through barrel end wall 776 of barrel component 754. For example, as shown in FIG. 15A, barrel end wall 776 can comprise first opening 792 and first groove 794. An end of a pull wire can be inserted into first opening 792 and secured therein by a knot, fastener, weld or the like. In examples, the pull wire can be wrapped around knob shaft 758 and secured thereto or looped around knob shaft 758. First groove 794 can extend from first opening 792 to provide a path for the pull wire that gradually joins the exterior of barrel end wall 776 to prevent kinking and the like. A second opening and groove can be provided on barrel end wall 776 opposite of first opening 792 and first groove 794. As such a pair of pull wires can be attached to and anchored on barrel end wall 776. In operation, as explained in greater detail below, barrel component 754 can be rotated by knob component 752 induce pushing and pulling on the pull wires connected to barrel end wall 776. In examples, barrel component 754 can comprise first pulley 560A or second pulley 560B of FIG. 11 or any of the wheels described herein, such as wheel 318 of FIG. 6A, wheel 612 of FIG. 12A and the like.

Rotatable barrel tensioning mechanism 750 can be configured to operate with knob component 752 and barrel component 754 to take up slack within the pull wires before pushing and pulling of the pull wires occurs, as discussed with reference to FIGS. 15F-15H.

Clutch disk 760 can be positioned inside of barrel end wall 776 so that clutch 768 faces barrel side wall 778. Clutch 768 can abut barrel side wall 778 outside of clutch socket 784. In examples, clutch 768 can abut stop 790 on the opposite side of clutch socket 784. Configured as such, knob component 752 can be initially rotated relative to barrel component 754 in only one direction.

Post 780 can be attached to a housing of an endoscope control handle, such as handle housing 606 of FIG. 12A, handle housing 656 of FIG. 13A, handle housing 706 of FIG. 14A and handle housing 806 of FIG. 16A. In examples, post 780 can extend through a bore or opening in the housing and base 782 can be located outside of the housing. In examples, post 780 and knob shaft 758 can extend coaxially. Thus, barrel component 754 can be rotatable relative to the housing. Thus, base 782 can act as another control knob. In other examples, base 782 can be rotatably mounted within the housing.

FIG. 15F is a perspective view of rotatable barrel tensioning mechanism 750 of FIG. 15A in a standby state. The configuration of FIG. 15F can be the configuration of rotatable barrel tensioning mechanism 750 as removed from packaging before use. As such, pull wires attached to barrel end wall 776 can have slack via the mechanism described herein. Pull wire 796 can initially include slack 798. In examples, pull wire 796 can be fed into first groove 794 for securing to knob shaft 758.

Knob 762 can be grasped by a user to rotate knob disk 756. As shown in FIG. 15F, knob disk 756 can be rotated clockwise so that extension 774 disengages from stop 790 and slides along barrel side wall 778 in a clockwise arcuate manner. As arranged in FIG. 15F, knob component 752 can be rotated nearly a full revolution before extension 774 is positioned adjacent clutch socket 784 on the opposite side of stop 790.

FIG. 15G is a perspective view of rotatable barrel tensioning mechanism 750 of FIG. 15A in an intermediate state after rotation through an arc. As shown in FIG. 15G, slack 798 can be removed from pull wire 796. From the position of FIG. 15G, knob component 752 can be further rotated so that extension 774 is positioned over extension portion 788 of clutch socket 784. When positioned as shown in FIG. 15G, spring 770 can push barrel component 754 upward relative to FIG. 15G. Thus, barrel component 754 can move away from base 782 and toward knob disk 756. Such upward movement of barrel component 754 can cause clutch socket 784 to be positioned over extension 774, e.g., extension 774 is moved into clutch socket 784. As such, barrel component 754 can become rotatably engaged with knob component 752.

FIG. 15H is a perspective view of rotatable barrel tensioning mechanism 750 of FIG. 15A in an activated state. Knob component 752 can continue to be rotated in the clockwise direction. Engagement of extension 774 with clutch socket 784 will cause barrel component 754 to rotate with knob component 752. In such a state, rotation of knob component 752 can induce pushing and pulling of pull wires connected to barrel end wall 776 with slack 798 removed from pull wire 796.

FIG. 16A is a schematic cross-sectional view of rack and pinion tensioning mechanism 800 for pull wires 802A and 802B in a standby state. FIG. 16B is a schematic cross-sectional view of the rack and pinion tensioning mechanism 800 of FIG. 16A in an activated state. FIGS. 14A and 14B are discussed concurrently.

Rack and pinion tensioning mechanism 800 can be used in endoscope controller 804. Endoscope controller 804 can comprise handle housing 806 and shaft 808. Handle housing 806 can be configured similarly as handle section 202 of FIG. 3 and shaft 808 can be configured similarly as shaft 204 of FIG. 3. Shaft 808 and handle housing 806 can be connected to each other in a fixed manner or can be connected to each other in a sliding manner as described with reference to FIGS. 3-5.

Pull wires 802A and 802B can extend from shaft 808 can be connected to actuator 810. Actuator 810 can comprise wheel 812, lever 814 and knob 816. In examples, pull wires 802A and 802B can comprise opposite end portions of a single wire wrapped around wheel 812. In examples, pull wires 802A and 802B can comprise two different wires connected to wheel 812.

Rack and pinion tensioning mechanism 800 can be disposed within or on handle housing 806 to interact with pull wires 802A and 802B. Rack and pinion tensioning mechanism 800 can comprise rack 824, pinion 826, shaft 828, spring 830 and actuator 832.

As discussed herein, endoscope controller 804 can be subject to manufacturing and environmental conditions that can introduce slack into pull wires 802A and 802B. As such, pull wires 802A and 802B can include slack 822A and 822B. Rack and pinion tensioning mechanism 800 can be configured to eliminate or reduce slack 822A and 822B from pull wires 802A and 802B, respectively.

Pull wires 802A and 802B can be fixed at the distal tip of shaft 808. Pull wires 802A and 802B can be sized, e.g., have a length, to accommodate a particular distance between actuator 810 and the distal tip of shaft 808, accommodating any fixed obstacles with handle housing 806. Thus, as shaft 808 changes shape, e.g., becomes reduced in length, proximal end face 834 of shaft 808 can become closer to actuator 810 as compared to before shaft 808 changed shape, resulting in the generation of slack. In examples, shaft 808 can shrink, thereby shortening in length so that pull wires 802A and 802B are longer than desirable to produce a change in the position of the distal tip of shaft 808 when the actuator is displaced, e.g., longer than desirable to produce no or very little knob dwell. In short, pull wires 802A and 802B can be longer than needed to extend between actuator 810 and the distal tip of shaft 808.

Rack and pinion tensioning mechanism 800 can be configured to push or pull on pull wires 802A and 802B to increase the length that pull wires must travel to reach the distal tip of shaft 808 from actuator 810 to remove slack 822A and 822B from pull wire 802B. Rack and pinion tensioning mechanism 800 can be configured so that rack 824 is in a first position to not have an effect on the tension of pull wires 802A and 802B, as shown in FIG. 16A. However, rack 824 can be advanced, e.g., linearly translated, away from actuator 810 so that slack 822A and 822B are removed from pull wires 802A and 802B, as shown in FIG. 16B.

Rack 824 can comprise an elongate body having a first end connected to proximal end face 834 of shaft 808 and a second end extending therefrom. In examples, rack 824 can be cantilevered from shaft 808. Rack 824 can be rigidly connected to shaft 808 and can comprise a rigid body such that rack 824 is configured to not move relative to shaft 808. The outward facing surface of rack 824 can include gear teeth to engage with pinion 826. Pinion 826 can comprise a circular gear having gear teeth configured to mesh with gear teeth of rack 824. Pinion 826 can be mounted to shaft 828 by any suitable means, such as a pinned connection. Pinion 826 can be fixed to shaft 828 such that relative rotation therebetween is prevented. Spring 830 can be connected to shaft 828 and can be configured to bias shaft 828 to advance rack 824 to a distal position. In examples, shaft 828 can comprise a coil spring or torsion spring configured to induce rotation in shaft 828. Spring 830 can be connected to actuator 832. In the standby state of FIG. 16A, actuator 832 can be positioned to immobilize, e.g., prevent rotation of, shaft 808 and hold rack 824 in a proximal position. In the activated state of FIG. 16B, actuator 832 to be repositioned to disengage from shaft 808 to thereby allow spring 830 to induce rotation in shaft 808. Actuator 832 can comprise a pin configured to engage shaft 828 that can be withdrawn my movement of a lever extending through handle housing 806. Rotation of shaft 808 from spring 830 can cause rotation of pinion 826, which can result in gear teeth of pinion 826 pushing gear teeth of rack 824 to cause translation of rack 824 away from handle housing 806. Shaft 808 can thereby be driven by rack 824 away from handle housing 806. Movement of shaft 808 away from handle housing 806 can lengthen the distance between shaft 808 and actuator 810, thereby removing slack 822A and 822B of pull wires 802A and 802B.

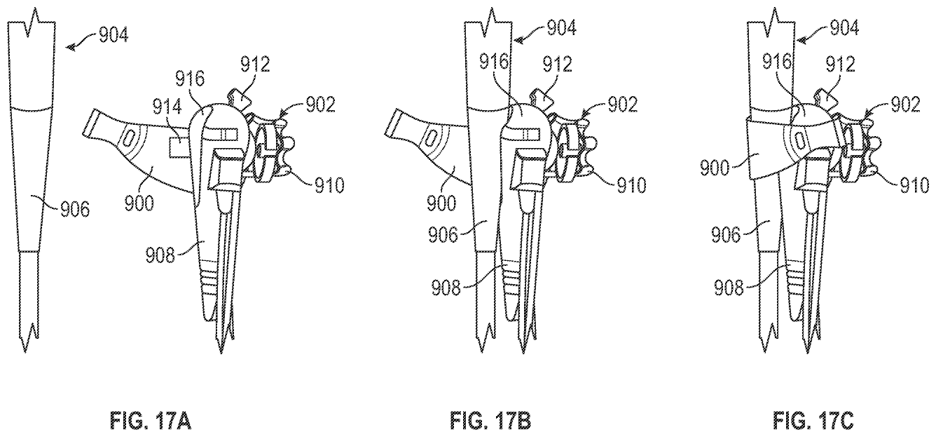

FIGS. 17A, 17B and 17C show retention strap 900 for auxiliary scope 902 being wrapped around primary scope 904 to actuate a tensioning mechanism of the present disclosure. Primary scope 904 can include shaft portion 906 that can extend between a proximal control handle and a distal working shaft. Auxiliary scope 902 can comprise handle 908 to which retention strap 900 can be attached. Handle 908 can include actuator 910 that can be used to activate a pull wire tensioning mechanism as described herein, lever 912 for operating features of auxiliary scope 902 and actuator 914 that can be used to activate a pull wire tensioning mechanism as described herein. In examples, actuator 914 can comprise button 618 of FIGS. 12A and 12B, actuation mechanism 719 of FIGS. 14A and 14B, actuator 832 of FIGS. 16A and 16B and features for the other tensioning mechanisms described herein.

In order to facilitate simultaneous or contemporaneous operation of auxiliary scope 902 with primary scope 904, auxiliary scope 902 can be attached to primary scope 904 in close proximity to the proximal control handle of primary scope 904 to facilitate two handed operation, with one hand on each of primary scope 904 and auxiliary scope 902 or to facilitate ready movement of one hand between primary scope 904 and auxiliary scope 902. Retention strap 900 can be used to secure auxiliary scope 902 to primary scope 904. Handle 908 of auxiliary scope 902 can include groove 916 into which shaft portion 906 of primary scope 904 can be placed. Positioning of shaft portion 906 into groove 916 can cause complete or partial activation of actuator 914. For example, actuator 914 can be displaced, e.g., depressed by the presence of shaft portion 906 in groove 916. Displacement of actuator 914 can cause the release of a tensioning mechanism as described herein. With shaft portion 906 in groove 916, retention strap 900 can be placed across shaft portion 906 and secured to handle 908 via suitable means, such as hook and loop fastener material, a snap or button or a fastener. In examples, securing of the free end of retention strap 900 to handle 908 can cause full displacement of actuator 914 to cause activation of a tensioning mechanism as described herein. As such, a user of auxiliary scope 902 need not actively or knowingly remove slack from pull wires, and the slack can be removed automatically by actuator 914 when auxiliary scope 902 is attached to primary scope 904.

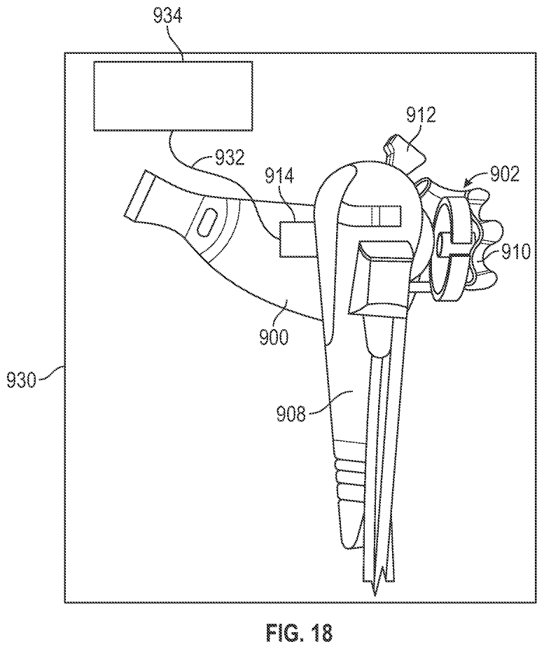

FIG. 18 is a schematic illustration of auxiliary scope 902 of FIGS. 17A-17B positioned within packaging container 930. Packaging container 930 can include tether 932 and anchor 934. Tether 932 can include a first end attached to actuator 914 and a second end attached to anchor 934. Anchor 934 can be attached to packaging container 930 in a fixed manner. Tether 932 can comprise a flexible cord or wire extending between actuator 914 and anchor 934. In examples, tether 932 can be long enough to include slack when handle 908 is positioned within packaging container 930. Thus, handle 908 can be removed from packaging container 930 a distance before tether 932 becomes taught. As handle 908 is pulled away from packaging container 930, tether 932 can become taught before activation of actuator 914. Pulling of handle 908 away from packaging container 930 to a distance greater than the length of tether 932 at a sufficient force can cause actuator 914 to activate. In examples, tether 932 can be attached in a break-away fashion. In examples, tether 932 can be attached to actuator 914 in a break-away manner such that the force required to break-away from actuator 914 can be greater than the force required to activate actuator 914. Thus, a movement of handle 908 away from packaging container 930 sufficient to cause separation between actuator 914 and tether 932 will first cause activation of actuator 914, followed by detachment of tether 932 from anchor 934.

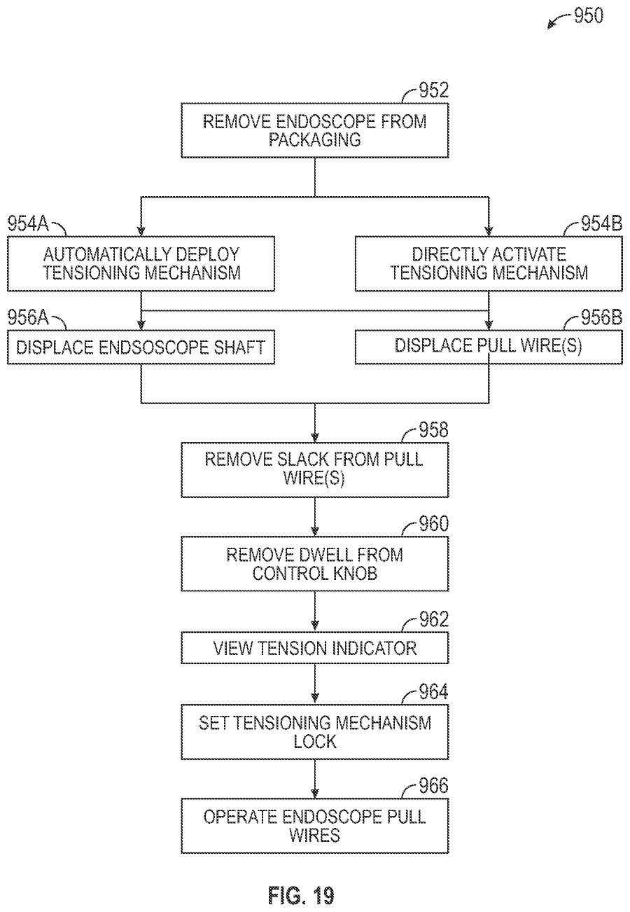

FIG. 19 is a block diagram illustrating operations of methods 950 for reducing slack in pull wires to, for example, reduce knob dwell.

At operation 952, an endoscope can be removed from product packaging in preparation for use in performing a procedure. The endoscope can be removed from a wrapper or box and then removed from a tray that holds the endoscope in the wrapper or box. Preparation for performance of the procedure can additionally include attaching the endoscope to an additional endoscope, such as a primary scope or duodenoscope.

At operation 954A, a pull wire tensioning mechanism can be automatically deployed. Automatic deployment of the tensioning mechanism can occur by the endoscope being removed from the packaging, such as by a tether being tensioned to activate a tensioning mechanism. In examples, a tether can be tensioned to remove a pin. Automatic deployment can additionally occur by strapping of the endoscope to the additional endoscope in order to activate a tensioning mechanism by the process of attaching the endoscope to the additional scope to avoid the need for the user to perform another step dedicated to the tensioning mechanism.

At operation 954B, a pull wire tensioning mechanism can be directly or manually actuated. Direct or manual deployment can occur by the user by direct intervention, such as by pushing a button, rotating a knob, lever or strain relief, moving or removing a pin, and the like. Direct or manual actuation can include moving an activation mechanism from an off position to an on position, or can comprise moving the activation mechanism to one or more intermediary positions between off and on positions.

At operation 956A, the pull wire tensioning mechanism can be actuated by displacing an endoscope shaft. As discussed herein, the endoscope shaft can be displaced by the examples of FIGS. 3-11, 16A and 16B, as well as via equivalent and other types of tensioning mechanisms.

At operation 956B, the pull wire tensioning mechanism can be actuated by displacing the pull wires.

At operation 956B, the pull wire tensioning mechanism can be actuated by displacing the pull wires. As discussed herein, the pull wires can be displaced by the examples of FIGS. 12A-15H, as well as via equivalent and other types of tensioning mechanisms.

At operation 958, displacement of the endoscope shaft or pull wires can remove slack in the pull wires, such as by applying tension to the pull wires. As discussed herein, slack arising from manufacturing processes, sterilization processes and environmental conditions can be reduced or eliminated with the tensioning mechanism described herein.

At operation 960, knob dwell in an actuator for pulling on the pull wires can be reduced or removed by the tensioning of the pull wires at either of operations 956A and 956B. Removal or reduction of slack at operation 958 can remove or reduce knob dwell in an actuator for operating pull wires. As such, the removal or reduction of knob dwell can provide a use with more response operation of the endoscope.

At operation 962, a tension indicator can be viewed on the endoscope. A tension indicator can be used to provide a user with an indication of whether or not a tensioning mechanism should be activated or should be additionally actuated. For example, a tension indicator can provide an initial indication of slack within the pull wires, such as by providing a window through which the pull wires can be viewed. In examples, the pull wire tensioning mechanism can be placed proximate a gauge or indicator that shows how far a tensioning mechanism is to be moved, displaced or actuated to provide minimal, maximum or intermedial levels of tension in the pull wires. As such, a user can select a desired amount of tension to reduce slack to provide a desired amount of knob dwell or eliminate knob dwell.

At operation 964, the pull wire tensioning mechanism can be locked to prevent recurrence of pull wire slack. For example, detents, latches, straps or stops can be employed to hold a tensioning mechanism in an advanced, activated or actuated state to hold the pull wires at the desired level of tension. Such tensioning mechanism locks can be automatically deployed without user action or can be manually deployed by a user.

At operation 966, the endoscope can be operated using the pull wire actuator. One the pull wire tensioning mechanism has be moved to the desired setting and locked in place, a suer can operate the endoscope to perform a medical procedure with the selected amount of knob dwell.

As discussed herein, the present disclosure is useful in providing mechanisms for tensioning pull wires of endoscope controllers, housing, handpieces and the like. Such pull wires can be used to provide articulation to, e.g., bending of, distal portions of endoscope shafts. However, the pull wires can become slack as a result of various manufacturing and sterilization procedures and exposure to environmental conditions. Application of tension to the pull wires can remove slack and knob dwell that results from slack. Additionally, the tensioning mechanisms can be used to allow a user to apply a desired amount of tension in pull wires to produce a desired amount of responsiveness. Gauges or indicia can be used to assist a user in determining if slack has been removed. The pull wire tensioning mechanisms can be automatically deployed in the course of preparing an endoscope for use, e.g., removing from packaging, rotation of a pull wire actuation mechanism in the normal course of action, or attaching the endoscope to another instrument, such as a primary scope. The pull wire tensioning mechanisms can be manually or directly deployed by removal of pins, pushing of buttons, rotating of knobs and levers and the like.

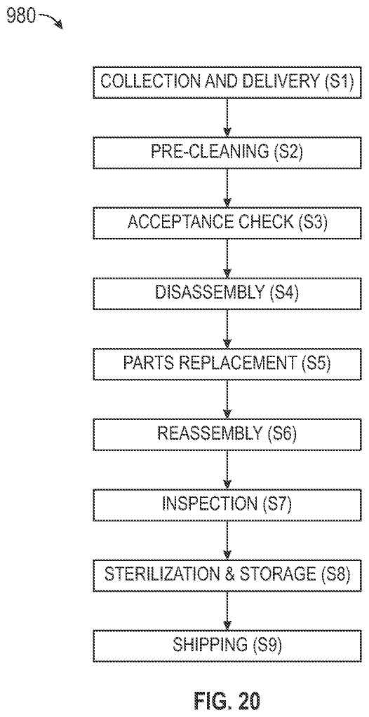

FIG. 20 is a flowchart indicating reprocessing method 980 for treatment instruments disclosed in the present application. The treatment instruments described above, such as controller 200 of FIG. 3, controller 306 of FIG. 6A, controller 374 of FIG. 7A, handle section 401 of FIG. 8, handle section 451 of FIG. 9, handle section 501 of FIG. 10A, handle section 551 of FIG. 11, controller 604 of FIG. 12A, controller 654 of FIG. 13A, controller 704 of FIG. 14A, rotatable barrel tensioning mechanism 750 of FIG. 15A and controller 804 of FIG. 16A, as well as the insertion sections and working shafts that can be attached thereto, may be disposed of after one use, or may be repeatedly used a plurality of times. In the case of a configuration that is repeatedly used a plurality of times, for example, the reprocessing method shown in FIG. 20 is required or can be used. An operator collects the used treatment instrument after the treatment instrument has been used for treatment and transports the treatment instrument to a factory or the like (Step S1). Then, the operator cleans and sterilizes the collected and transported used treatment instrument (Step S2). Next, the operator performs an acceptance check of the used treatment instrument (Step S3). Subsequently, the operator disassembles the used treatment instrument (Step S4) and replaces some parts of the used treatment instrument with new parts (Step S5). After step S5, the operator assembles a new or reprocessed treatment instrument (Step S6). In some examples, Step S6 can include adding an identifier to indicate the device has been modified from its original condition, such as a adding a label or other marking to designate the device as reprocessed, refurbished or remanufactured. After Step S6, the operator sequentially performs an inspection (Step S7), sterilization and storage (Step S8), and shipping (Step S9) of the new treatment instrument. The treatment instruments according to the present embodiments have tensioning mechanisms that can be reprocessed for multiple uses. Therefore, there is advantage that the tensioning mechanisms of the present disclosure can reduce the cost of medical procedures.

### EXAMPLES

Example 1 is an endoscope comprising: a handpiece housing; an elongate flexible shaft extending from the handpiece housing; a pull wire extending from the handpiece housing into the elongate flexible shaft; and a tensioning mechanism configured to adjust tension in the pull wire.

In Example 2, the subject matter of Example 1 optionally includes wherein the tensioning mechanism is configured to adjust a position of the elongate flexible shaft relative to the handpiece housing to indirectly adjust tension in the pull wire.

In Example 3, the subject matter of Example 2 optionally includes wherein the tensioning mechanism comprises a rotatable hub positioned around the elongate flexible shaft that is configured to induce axial translation of the elongate flexible shaft.

In Example 4, the subject matter of Example 3 optionally includes wherein the rotatable hub is positioned outside of the handpiece housing and is connected to a rotatable strain relief positioned around the elongate flexible shaft.

In Example 5, the subject matter of any one or more of Examples 3-4 optionally include wherein the rotatable hub is positioned inside of the handpiece housing and is connected to a lever extending through the handpiece housing.

In Example 6, the subject matter of any one or more of Examples 3-5 optionally include wherein: the rotatable hub comprises a spiral slot; and the elongate flexible shaft comprises a pin configured to ride in the spiral slot.

In Example 7, the subject matter of any one or more of Examples 2-6 optionally include wherein the elongate flexible shaft is connected to a guide component to maintain the elongate flexible shaft aligned within the handpiece housing.

In Example 8, the subject matter of any one or more of Examples 2-7 optionally include wherein the tensioning mechanism comprises a spring-activated device.

In Example 9, the subject matter of Example 8 optionally includes wherein the spring-activated device comprises: a shuttle connected to the elongate flexible shaft; a guide body of the handpiece housing in which the shuttle is configured to slide; and a spring connected to the guide body to push against the shuttle.

In Example 10, the subject matter of any one or more of Examples 8-9 optionally include wherein the spring-activated device comprises: a stop wall of the handpiece housing; a proximal surface of the elongate flexible shaft; a spring disposed between the proximal surface and the stop wall; and a pin configured to hold the spring in a compressed state, the pin being displaceable relative to the handpiece housing to allow the spring to expand.

In Example 11, the subject matter of any one or more of Examples 2-10 optionally include wherein the tensioning mechanism comprises a ratchet mechanism.

In Example 12, the subject matter of Example 11 optionally includes wherein the ratchet mechanism comprises: a rail extending form the handpiece housing; a ratchet block connected to the elongate flexible shaft, the ratchet block comprising: a slot to receive the rail; and a lever extending from the ratchet block to a location exterior of the handpiece housing, the lever including lever teeth; and a ratchet strip having ratchet teeth configured to engage the lever teeth.

In Example 13, the subject matter of any one or more of Examples 2-12 optionally include wherein the tensioning mechanism comprises a screw mechanism.

In Example 14, the subject matter of Example 13 optionally includes wherein the screw mechanism comprises: a rail extending form the handpiece housing; a slide body comprising a slot to engage the rail; a screw body extending from the slide body and connected to the elongate flexible shaft; and a knob positioned around the screw body to extend at least partially through a window in the handpiece housing.

In Example 15, the subject matter of any one or more of Examples 2-14 optionally include wherein the tensioning mechanism comprises: a button extending into the handpiece housing; and a wedge connected to the elongate flexible shaft, the button configured to engage the wedge to axially displace the elongate flexible shaft.

In Example 16, the subject matter of any one or more of Examples 2-15 optionally include wherein the tensioning mechanism comprises a rack and pinion tensioning mechanism configured to displace the elongate flexible shaft.

In Example 17, the subject matter of Example 16 optionally includes wherein the rack and pinion tensioning mechanism comprises: a rack gear connected to the elongate flexible shaft; a pinion gear mounted on a shaft to engage the rack gear; a torsion spring configured to bias the shaft; and an actuator configured to selectively release compression of the torsion spring to rotate the pinion gear to drive the rack gear.

In Example 18, the subject matter of any one or more of Examples 2-17 optionally include a locking mechanism to maintain the elongate flexible shaft in a distal position after the tensioning mechanism has been engaged.

In Example 19, the subject matter of any one or more of Examples 1-18 optionally include a release mechanism configured to actuate the tensioning mechanism when the endoscope is removed from a packaging component.

In Example 20, the subject matter of Example 19 optionally includes wherein the release mechanism comprises a tether attached to the tensioning mechanism to activate the tensioning mechanism.

In Example 21, the subject matter of any one or more of Examples 1-20 optionally include an actuation mechanism connected to the pull wire to pull on the pull wire independently of the tensioning mechanism.

In Example 22, the subject matter of any one or more of Examples 1-21 optionally include wherein the tensioning mechanism is configured displace the pull wire to increase a distance traveled by the pull wire to directly adjust tension in the pull wire.

In Example 23, the subject matter of any one or more of Examples 1-22 optionally include wherein the tensioning mechanism comprises an indicator.

In Example 24, the subject matter of Example 23 optionally includes wherein the tensioning mechanism comprises a spring-loaded tensioning mechanism configured to displace the pull wire.

In Example 25, the subject matter of Example 24 optionally includes wherein the spring-loaded tensioning mechanism comprises: a tensioner configured to engage the pull wire; a spring configured to push against the tensioner; an actuation mechanism configured to hold the spring in a compressed state; and a button accessible from an exterior of the handpiece housing to release the actuation mechanism.

In Example 26, the subject matter of any one or more of Examples 23-25 optionally include wherein the tensioning mechanism comprises a torsion-spring tensioning mechanism configured to wind the pull wire.

In Example 27, the subject matter of Example 26 optionally includes wherein the torsion-spring tensioning mechanism comprises: a drum mounted in the handpiece housing on a pin forming a rotation axis; a slot configured to receive the pin to allow the drum to move axially relative to the handpiece housing; and a torsion spring connecting the drum to the pin; wherein the pull wire is configured to at least partially wrap around the drum.

In Example 28, the subject matter of any one or more of Examples 23-27 optionally include wherein the tensioning mechanism comprises a rotatable-drum tensioning mechanism configured to displace the pull wire.

In Example 29, the subject matter of Example 28 optionally includes wherein the rotatable-drum tensioning mechanism comprises: a drum mounted in the handpiece housing to rotate on an axis extending parallel to the pull wire; a tensioner extending from the drum to engage the pull wire; and a lever connected to the drum to rotate the drum along the axis to displace the pull wire; wherein the tensioner is configured to displace the pull wire radially.

In Example 30, the subject matter of any one or more of Examples 23-29 optionally include wherein the tensioning mechanism comprises a slack-take up barrel incorporated into an actuation mechanism for the pull wire.

In Example 31, the subject matter of Example 30 optionally includes wherein the slack-take up barrel comprises: a barrel rotatable on a first axis perpendicular to the pull wire; a knob rotatable on a second axis coaxial with the first axis; and a clutch mechanism configured to allow rotation of the knob relative to the barrel over an arc before the knob locks relative to the barrel; wherein the pull wire is configured to at least partially wrap around the barrel.

Example 32 is a method for adjusting tension in pull wires of a controller for an endoscope, the method comprising: preparing an endoscope for use before a procedure; adjusting tension in a pull wire for deflecting a flexible elongate shaft of the endoscope to reduce knob dwell; and performing an endoscopic procedure with the endoscope.

In Example 33, the subject matter of Example 32 optionally includes wherein preparing the endoscope for use before the procedure comprises removing the endoscope from packaging.

In Example 34, the subject matter of Example 33 optionally includes wherein adjusting tension in the pull wire for deflecting the flexible elongate shaft of the endoscope to reduce knob dwell comprises activating a tensioning mechanism to adjust the tension in the pull wire by removing the endoscope from the packaging.

In Example 35, the subject matter of Example 34 optionally includes wherein activating the tensioning mechanism comprises pulling a pin from the tensioning mechanism with a tether.

In Example 36, the subject matter of any one or more of Examples 32-35 optionally include wherein adjusting tension in the pull wire for deflecting the flexible elongate shaft of the endoscope to reduce knob dwell comprises displacing the flexible elongate shaft.

In Example 37, the subject matter of Example 36 optionally includes wherein displacing the flexible elongate shaft comprises rotating a helical tensioning mechanism.

In Example 38, the subject matter of any one or more of Examples 36-37 optionally include wherein displacing the flexible elongate shaft comprises releasing a spring-activated tensioning mechanism.

In Example 39, the subject matter of any one or more of Examples 36-38 optionally include wherein displacing the flexible elongate shaft comprises rotating a screw-activated tensioning mechanism.

In Example 40, the subject matter of any one or more of Examples 36-39 optionally include wherein displacing the flexible elongate shaft comprises operating a rack and pinion tensioning mechanism.

In Example 41, the subject matter of any one or more of Examples 32-40 optionally include viewing a tensioning mechanism gauge that indicates a tension level.

In Example 42, the subject matter of any one or more of Examples 32-41 optionally include setting a tensioning mechanism lock to hold the tension in the pull wire.

In Example 43, the subject matter of any one or more of Examples 32-42 optionally include operating the endoscope with a reduced level of knob dwell as compared to before the endoscope was prepared for use before the procedure.

In Example 44, the subject matter of any one or more of Examples 32-43 optionally include wherein adjusting tension in the pull wire for deflecting the flexible elongate shaft of the endoscope to reduce knob dwell comprises displacing the pull wire.

In Example 45, the subject matter of any one or more of Examples 32-44 optionally include wherein displacing the pull wire comprises engaging a spring-loaded tensioner with the pull wire to displace the pull wire radially relative to an axial direction of the pull wire.

In Example 46, the subject matter of any one or more of Examples 32-45 optionally include wherein displacing the pull wire comprises rotating the pull wire about a drum to increase a travel path of the pull wire.

In Example 47, the subject matter of any one or more of Examples 32-46 optionally include wherein displacing the pull wire comprises winding the pull wire within a drum to cinch the pull wire.

In Example 48, the subject matter of any one or more of Examples 32-47 optionally include wherein displacing the pull wire comprises winding slack of the pull wire within an actuator for pulling the pull wire to deflect the flexible elongate shaft of the endoscope.

In Example 49, the subject matter of any one or more of Examples 32-48 optionally include wherein preparing the endoscope for use comprises attaching the endoscope to an additional endoscope.

In Example 50, the subject matter of Example 49 optionally includes wherein adjusting tension in the pull wire for deflecting the flexible elongate shaft of the endoscope to reduce knob dwell comprises activating an actuator for a tensioning mechanism to adjust the tension in the pull wire by strapping the endoscope to the additional endoscope.

Each of these non-limiting examples can stand on its own, or can be combined in various permutations or combinations with one or more of the other examples.

### Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. An endoscope comprising:
a handpiece housing;
an elongate flexible shaft extending from the handpiece housing;
a pull wire extending from the handpiece housing into the elongate flexible shaft; and
a tensioning mechanism configured to adjust tension in the pull wire.

2. The endoscope of claim 1, wherein the tensioning mechanism is configured to adjust a position of the elongate flexible shaft relative to the handpiece housing to indirectly adjust tension in the pull wire.

3. The endoscope of claim 2, wherein the tensioning mechanism comprises a rotatable hub positioned around the elongate flexible shaft that is configured to induce axial translation of the elongate flexible shaft.

4. The endoscope of claim 3, wherein the rotatable hub is positioned outside of the handpiece housing and is connected to a rotatable strain relief positioned around the elongate flexible shaft.

5. The endoscope of claim 3, wherein the rotatable hub is positioned inside of the handpiece housing and is connected to a lever extending through the handpiece housing, wherein:
the rotatable hub comprises a spiral slot; and
the elongate flexible shaft comprises a pin configured to ride in the spiral slot.

6. The endoscope of claim 2, wherein the tensioning mechanism comprises a spring-activated device.

7. The endoscope of claim 6, wherein the spring-activated device comprises:
a shuttle connected to the elongate flexible shaft;
a guide body of the handpiece housing in which the shuttle is configured to slide; and
a spring connected to the guide body to push against the shuttle.

8. The endoscope of claim 6, wherein the spring-activated device comprises:
a stop wall of the handpiece housing;
a proximal surface of the elongate flexible shaft;
a spring disposed between the proximal surface and the stop wall; and
a pin configured to hold the spring in a compressed state, the pin being displaceable relative to the handpiece housing to allow the spring to expand.

9. The endoscope of claim 2, wherein the tensioning mechanism comprises a ratchet mechanism, wherein the ratchet mechanism comprises:
a rail extending form the handpiece housing;
a ratchet block connected to the elongate flexible shaft, the ratchet block comprising:
a slot to receive the rail; and
a lever extending from the ratchet block to a location exterior of the handpiece housing, the lever including lever teeth; and
a ratchet strip having ratchet teeth configured to engage the lever teeth.

10. The endoscope of claim 2, wherein the tensioning mechanism comprises a screw mechanism, wherein the screw mechanism comprises:
a rail extending form the handpiece housing;
a slide body comprising a slot to engage the rail;
a screw body extending from the slide body and connected to the elongate flexible shaft; and
a knob positioned around the screw body to extend at least partially through a window in the handpiece housing.

11. The endoscope of claim 2, wherein the tensioning mechanism comprises:
a button extending into the handpiece housing; and
a wedge connected to the elongate flexible shaft, the button configured to engage the wedge to axially displace the elongate flexible shaft.

12. The endoscope of claim 2, wherein the tensioning mechanism comprises a rack and pinion tensioning mechanism configured to displace the elongate flexible shaft, wherein the rack and pinion tensioning mechanism comprises:
a rack gear connected to the elongate flexible shaft;
a pinion gear mounted on a shaft to engage the rack gear;
a torsion spring configured to bias the shaft; and
an actuator configured to selectively release compression of the torsion spring to rotate the pinion gear to drive the rack gear.

13. The endoscope of claim 2, further comprising a locking mechanism to maintain the elongate flexible shaft in a distal position after the tensioning mechanism has been engaged.

14. The endoscope of claim 1, further comprising a release mechanism configured to actuate the tensioning mechanism when the endoscope is removed from a packaging component, wherein the release mechanism comprises a tether attached to the tensioning mechanism to activate the tensioning mechanism.

15. The endoscope of claim 1, further comprising an actuation mechanism connected to the pull wire to pull on the pull wire independently of the tensioning mechanism.

16. The endoscope of claim 1, wherein the tensioning mechanism is configured displace the pull wire to increase a distance traveled by the pull wire to directly adjust tension in the pull wire.

17. The endoscope of claim 16, wherein the tensioning mechanism comprises a spring-loaded tensioning mechanism configured to displace the pull wire, wherein the spring-loaded tensioning mechanism comprises:
a tensioner configured to engage the pull wire;
a spring configured to push against the tensioner;
an actuation mechanism configured to hold the spring in a compressed state; and
a button accessible from an exterior of the handpiece housing to release the actuation mechanism.

18. The endoscope of claim 16, wherein the tensioning mechanism comprises a torsion-spring tensioning mechanism configured to wind the pull wire, wherein the torsion-spring tensioning mechanism comprises:
a drum mounted in the handpiece housing on a pin forming a rotation axis;
a slot configured to receive the pin to allow the drum to move axially relative to the handpiece housing; and
a torsion spring connecting the drum to the pin;
wherein the pull wire is configured to at least partially wrap around the drum.

19. The endoscope of claim 16, wherein the tensioning mechanism comprises a rotatable-drum tensioning mechanism configured to displace the pull wire, wherein the rotatable-drum tensioning mechanism comprises:
a drum mounted in the handpiece housing to rotate on an axis extending parallel to the pull wire;
a tensioner extending from the drum to engage the pull wire; and
a lever connected to the drum to rotate the drum along the axis to displace the pull wire;
wherein the tensioner is configured to displace the pull wire radially.

20. The endoscope of claim 16, wherein the tensioning mechanism comprises a slack-take up barrel incorporated into an actuation mechanism for the pull wire, wherein the slack-take up barrel comprises:
a barrel rotatable on a first axis perpendicular to the pull wire;
a knob rotatable on a second axis coaxial with the first axis; and
a clutch mechanism configured to allow rotation of the knob relative to the barrel over an arc before the knob locks relative to the barrel;
wherein the pull wire is configured to at least partially wrap around the barrel.

21. A method for adjusting tension in pull wires of a controller for an endoscope, the method comprising:
preparing an endoscope for use before a procedure;
adjusting tension in a pull wire for deflecting a flexible elongate shaft of the endoscope to reduce knob dwell; and
performing an endoscopic procedure with the endoscope.

22. The method of claim 21, wherein:
preparing the endoscope for use before the procedure comprises removing the endoscope from packaging; and
adjusting tension in the pull wire for deflecting the flexible elongate shaft of the endoscope to reduce knob dwell comprises activating a tensioning mechanism to adjust the tension in the pull wire by removing the endoscope from the packaging, wherein activating the tensioning mechanism comprises pulling a pin from the tensioning mechanism with a tether.

23. The method of claim 21, wherein adjusting tension in the pull wire for deflecting the flexible elongate shaft of the endoscope to reduce knob dwell comprises displacing the flexible elongate shaft.

24. The method of claim 23, wherein displacing the flexible elongate shaft comprises rotating a helical tensioning mechanism.

25. The method of claim 23, wherein displacing the flexible elongate shaft comprises releasing a spring-activated tensioning mechanism.

26. The method of claim 23, wherein displacing the flexible elongate shaft comprises rotating a screw-activated tensioning mechanism.

27. The method of claim 23, wherein displacing the flexible elongate shaft comprises operating a rack and pinion tensioning mechanism.

28. The method of claim 21, further comprising setting a tensioning mechanism lock to hold the tension in the pull wire.

29. The method of claim 21, wherein adjusting tension in the pull wire for deflecting the flexible elongate shaft of the endoscope to reduce knob dwell comprises displacing the pull wire.

30. The method of claim 29, wherein displacing the pull wire comprises engaging a spring-loaded tensioner with the pull wire to displace the pull wire radially relative to an axial direction of the pull wire.

31. The method of claim 29, wherein displacing the pull wire comprises rotating the pull wire about a drum to increase a travel path of the pull wire.

32. The method of claim 29, wherein displacing the pull wire comprises winding the pull wire within a drum to cinch the pull wire.

33. The method of claim 29, wherein displacing the pull wire comprises winding slack of the pull wire within an actuator for pulling the pull wire to deflect the flexible elongate shaft of the endoscope.

34. The method of claim 21, wherein preparing the endoscope for use comprises attaching the endoscope to an additional endoscope, wherein adjusting tension in the pull wire for deflecting the flexible elongate shaft of the endoscope to reduce knob dwell comprises activating an actuator for a tensioning mechanism to adjust the tension in the pull wire by strapping the endoscope to the additional endoscope.
